(19) United States
(12) Patent Application Publication
(10) Pub. No.: US 2010/0273895 A1
(43) Pub. Date: Oct. 28, 2010

(54) FORMULATIONS OF CANNABIDIOL AND PRODRUGS OF CANNABIDIOL AND METHODS OF USING THE SAME

(75) Inventors: Audra Lynn Stinchcomb, Lexington, KY (US); Stan Lee Banks, Frankfort, KY (US)

Correspondence Address: MAYER BROWN LLP, P.O. BOX 2828, CHICAGO, IL, 60690, US

(73) Assignee: AllTranz Inc., Lexington, KY (US)

(21) Appl. No.: 12/769,519

(22) Filed: Apr. 28, 2010

Related U.S. Application Data
(60) Provisional application No. 61/173,469, filed on Apr. 28, 2009.

(51) Int. Cl. A61K 31/05; A61P 1/00; A61P 29/00; A61P 1/08; A61P 25/22; A61P 25/08

(52) U.S. Cl. 514/733

(57) ABSTRACT

Described herein are pharmaceutical compositions comprising a cannabinoid, such as cannabidiol or a cannabidiol prodrug, which is metabolized to cannabidiol, and a penetration enhancer. Also described herein are methods of using the same. One embodiment described herein relates to the transdermal or topical administration of pharmaceutical compositions comprising a cannabinoid, such as cannabidiol or a cannabidiol prodrug, and a penetration enhancer to a person in need thereof.

### CROSS-REFERENCE TO RELATED APPLICATIONS

This application the benefit of U.S. Provisional Application No. 61/173,469, filed on Apr. 28, 2009, which is hereby incorporated by reference in its entirety to the extent permitted by law.

### FIELD

Described herein are compositions comprising pharmaceutically active cannabinoids, including cannabidiol and prodrugs of cannabidiol, suitable for local and systemic delivery to a mammal, which includes systemic transdermal delivery and topical delivery; and the use of such compositions in treating and preventing diseases and disorders, as well as improving cosmetic appearance.

### BACKGROUND

The clinical usefulness of cannabinoids, including cannabidiol (“CBD”), to provide analgesia and neuroprotection, reduce inflammation, help alleviate nausea and emesis, as well as treat epilepsy, anxiety disorders, and glaucoma, has been well-recognized. In addition, it is also well-known that cannabidiol lacks the psychoactive effects seen in many of the other cannabinoids, including Δ⁹-tetrahydrocannabinol, which is currently available in an oral dosage form, sold under the trade name Marinol®.

Pain is the most frequently reported symptom and it is a common clinical problem confronting all clinicians. Millions of people in the United States suffer from severe pain that, according to numerous recent reports, is chronically under-treated or inappropriately managed. Similarly, millions of people also suffer from severe nausea and/or frequent emesis. Moreover, all too frequently, many patients suffering from chronic, under-treated or irretraceable pain also suffer from lack of appetite, nausea and/or frequent emesis. These patients present a greater clinical challenge as they are unable to receive effective doses of oral pain medications, thereby leaving their pain unalleviated. Cannabinoids, including cannabidiol, are effective in alleviating pain. Moreover, cannabinoids, including cannabidiol, can reduce a patient's nausea and vomiting, independent of any pain relief achieved. Thus, cannabinoids are particularly useful in patients experiencing nausea and vomiting secondary to un- or under-treated pain.

A notable percentage of the United States population satisfy the diagnostic criteria for alcohol use disorders (“AUDs”). The consumption of excessive amounts of alcohol results in a complex array of pharmacological effects that directly impact the ability to treat the condition. These effects directly impact the brain and include progressive neurodegeneration, impaired executive function and dependence leading to withdrawal-induced negative effects. It is known that cannabinoids, including cannabidiol, have neuroprotective, anxiolytic and anti-convulsant effects, which may be effective in preventing additional brain damage in persons with AUDs, while simultaneously decreasing the frequency of relapses.

Chronic abusers of cannabis can develop dependence and experience withdrawal symptoms when they attempt to discontinue use of the drug. Collectively cannabis dependence and withdrawal are referred to herein as cannabis use disorders. It is known to those of skill in the art that cannabinoids, including cannabidiol, are useful in treating cannabis use disorders.

Dystonia is a neurological movement disorder, with many known causes, and characterized by involuntary, continual muscular contractions causing twisting and repetitive movements or abnormal postures. Cannabinoids have been shown to reduce the muscular contractions characteristic of this disorder.

The etiological pathology of many diseases relates to the inflammatory processes that are regulated by an individual's immune system. Inflammation may result from (1) an otherwise appropriate immunoresponse to an outside trauma, such as brain swelling secondary to a closed head injury; (2) an overactive immunoresponse, such as an allergic reaction or dermatitis; or (3) an inappropriate auto-immunoresponse, such as certain forms of multiple sclerosis, inflammatory bowel disorders and arthritis. Regardless of the underlying cause of the inflammation, it is therapeutically desirable under these circumstances to regulate the immune system and lessen the inflammatory response. Cannabinoids have been shown to regulate various steps in the immune response and could show some therapeutic benefit in the treatment of certain inflammatory diseases such as psoriatic arthritis.

Rheumatoid arthritis affects approximately 0.5-1% of the United States population, and autoimmune diseases in general affect more than 20 million Americans. The pain associated with rheumatoid arthritis can often be disabling. Cannabinoids have been found to be useful as an adjunct treatment for rheumatoid arthritis and joint pain secondary to other autoimmune diseases, such as inflammatory bowel disease, multiple sclerosis and systemic lupus erythematosus.

In addition to the above-discussed therapeutics benefits, cannabinoids, such as cannabidiol and cannabidiol prodrugs, present a variety of pharmacological benefits, including, but not limited to, anti-inflammatory, anti-convulsant, anti-psychotic, antioxidant, neuroprotective, anti-cancer and immunomodulatory effects.

Given these systemic therapeutic benefits, it would be advantageous to develop a composition in which cannabidiol is delivered systemically to achieve therapeutically effective plasma concentrations in a patient. However, cannabinoid oral dosage forms, including cannabidiol, must overcome several obstacles in order to achieve a systemic concentration. First, cannabinoids are generally highly lipophilic. Their limited water solubility thereby restricts the amount of cannabinoid available for absorption in the gastrointestinal tract. Second, cannabidiol, as with the other cannabinoids, undergoes substantial first-pass metabolism when absorbed from the human gastrointestinal tract. Finally, the oral bioavailability of any product is further diminished when a patient suffers from nausea or emesis, as either the patient avoids taking his oral medications or the oral dosage form does not remain in the gastrointestinal tract for a sufficient period of time to release the entire dose and achieve a therapeutic concentration.

Therefore, in view of the foregoing, it would be desirable to systemically deliver therapeutically effective amounts of cannabidiol to a mammal in need thereof for the treatment of one or more medical conditions responsive to cannabidiol, including pain, nausea or appetite stimulation, by a route of administration that does not depend upon absorption from the gastrointestinal tract of the mammal and is not subject to first-pass metabolism upon absorption from the gastrointestinal tract. One non-oral route of administration for the systemic delivery of cannabidiol is transdermal administration.

Unfortunately, due to its highly hydrophobic nature, cannabidiol is poorly absorbed through membranes such as the skin of mammals, including humans. Therefore, the success of transdermally administering therapeutically effective quantities of cannabidiol to a mammal in need of such treatment within a reasonable time frame and over a suitable surface area has been substantially limited. However, it has been found that the rate and extent of cannabidiol transdermal absorption can be improved by administering cannabidiol in compositions comprising penetration enhancers that improve absorption across the skin. It has further been discovered that by optimizing the excipients, the cannabidiol or cannabidiol prodrug can be administered on a schedule that encourages patient compliance, such as once or twice daily. Described herein are compositions comprising cannabinoids, including cannabidiol and penetration enhancers that when transdermally administered to a mammal, such as a human, provide a therapeutic systemic concentration of cannabidiol. Also described herein are methods of using compositions comprising penetration enhancers and cannabinoids, including cannabidiol.

In addition, the epidermis and dermis of many mammals, such as humans and guinea pigs, contains enzymes which are capable of metabolizing active pharmaceutical agents which pass through the stratum corneum. The metabolic process occurring in the skin of mammals, such as humans, can be utilized to deliver pharmaceutically effective quantities of cannabidiol to the systemic circulation of a mammal in need thereof. Described herein are prodrugs of cannabidiol and compositions comprising prodrugs of cannabidiol that can be transdermally administered to a mammal, such as a human, so that the metabolic product resulting from metabolism in the skin is cannabidiol which is systemically available for the treatment of a medical condition responsive to cannabidiol, including pain or nausea. Also described herein are compositions which may be suitable for transdermal delivery to a mammal and comprise penetration enhancers and cannabidiol prodrugs, wherein the metabolic product of the cannabidiol prodrug is cannabidiol, and when transdermally administered to a mammal, such as a human, may provide a therapeutically effective systemic concentration of cannabidiol. Also described herein are methods of using compositions comprising penetration enhancers and cannabidiol prodrugs, wherein the metabolic product of the cannabidiol prodrug is cannabidiol.

In addition to the enzymatic pathways occurring in the skin, other metabolic processes occurring in mammals, such as humans, can also be utilized to deliver pharmaceutically effective quantities of cannabidiol to the systemic circulation of a mammal in need thereof. Therefore, pharmaceutical compositions comprising cannabidiol prodrugs can be administered by other means, including: oral, buccal, ocular, sublingual, injection, rectal, vaginal and intranasal, to achieve a systemic therapeutically effective concentration. Administration by these means is advantageous because cannabidiol and cannabidiol prodrugs are generally well-absorbed by the membranes at these sites of administration. In addition, except when administered orally, administration by these means is favorable because, as with transdermal administration, first-pass metabolism is avoided. Therefore, a significant advancement in the art would occur with the development of a composition suitable for oral, buccal, sublingual, injectable, topical, follicular, nasal, ocular, rectal, vaginal delivery comprising cannabidiol or prodrugs of cannabidiol.

Described herein are compositions suitable for oral, buccal, sublingual, injectable, topical, follicular, nasal, ocular, rectal, vaginal delivery comprising cannabidiol or cannabidiol prodrugs, wherein the metabolic product of the cannabidiol prodrug is cannabidiol, that can be administered to a mammal, such as a human, whereby cannabidiol is available for the treatment of a medical condition responsive to cannabidiol, including as pain, nausea or appetite stimulation. Also described herein are methods of using compositions comprising cannabidiol or cannabidiol prodrugs, which are administered orally, buccally, sublingually, topically, follicularly, nasally, ocularly, rectally, vaginally and via injection.

In addition to the benefits of systemically administered cannabidiol discussed above, cannabinoids, including cannabidiol, have been found to have localized benefits from topical administration. For example, topically administered cannabinoids have been found to be useful to alleviate pain and other conditions originating at or near the surface of the skin, including but not limited to, pain associated with post-herpetic neuralgia, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis and psoriatic arthritis. In addition, topically administered cannabinoids have been found to be useful to alleviate pain and other conditions associated with deeper tissues, such as peripheral nerves, muscles and synovial tissues. Examples of conditions associated with deeper tissues responsive to cannabinoids include: peripheral neuropathic pain, including but not limited to the peripheral neuropathic pain associated with diabetic neuropathy, ankylosing spondylitis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid osteoarthritis, synovitis and juvenile rheumatoid arthritis. When cannabinoids are administered topically to treat pain and other conditions associated with deeper tissues, including peripheral neuropathic pain, it may be useful to co-administer cannabinoids systemically. Also, it has been found that the topical administration of cannabinoids, including cannabidiol, can inhibit the growth of hair.

In order to achieve these local benefits, it may be advantageous for cannabidiol or a prodrug thereof to penetrate the stratum corneum but not be absorbed systemically. In such a case, the cannabidiol would concentrate in the skin and/or pilosebaceous unit, thus maximizing its local effect. Not only does the localized effect increase the potential therapeutic benefit, it lessens the frequency and severity of side-effects associated with systemic cannabinoid administration because the amount of active compound circulating in the patient is reduced. The cannabidiol or cannabidiol prodrug can be incorporated into a formulation with an additional active moiety that is capable of improving the appearance and/or hydration of the skin.

Therefore, a significant advancement in the art would occur with the development of a composition suitable for topical delivery comprising cannabidiol or prodrugs of cannabidiol, wherein the resulting metabolic product of the cannabidiol prodrug is cannabidiol, whereby cannabidiol is available at the site of administration in a mammal in a therapeutically effective amount but is not absorbed systemically in a therapeutically effective concentration.

### SUMMARY

Described herein are compositions comprising cannabinoids, including cannabidiol and cannabidiol prodrugs and methods of using compositions comprising cannabinoids, including cannabidiol and prodrugs of cannabidiol.

Other embodiments, objects, features and advantages will be set forth in the detailed description of the embodiments that follows, and in part will be apparent from the description, or may be learned by practice, of the claimed invention. These objects and advantages will be realized and attained by the processes and compositions described and claimed herein. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### BRIEF DESCRIPTION OF THE DRAWINGS

FIG. 1 illustrates the cumulative permeation of 5% and 10% CBD gel over 24 hours with a single dose.

FIG. 2 illustrates the cumulative permeation of 5% CBD gel with varying concentrations of transcutol over 24 hours with a single dose.

FIG. 3 illustrates the cumulative permeation of 5% CBD gel and 7.5% transcutol with varying concentrations of propylene glycol (“PG”) over 24 hours with a single dose.

FIG. 4 illustrates the cumulative permeation of 5% CBD gel with 15% propylene glycol and varying concentrations of transcutol.

FIG. 5 illustrates the cumulative permeation of 5% CBD gel with 1.0% Carbopol, 2.0% Klucel and 1.5% Carbopol.

FIG. 6 illustrates the cumulative permeation of 2.5% CBD gel with 45% ethanol (“EtOH”), 5% CBD gel with 46% ethanol and 5% CBD gel with 54.5% ethanol.

FIG. 7 illustrates the cumulative permeation profile of 2.5% CBD gel dosed either twice daily or once daily for 3 days.

FIG. 8 illustrates the cumulative permeation profile of 2.5% CBD gel versus 4.0% CBD gel dosed twice daily.

FIG. 9 illustrates the cumulative permeation profile of 2.5% CBD gel versus 4.0% CBD gel (increased Carbopol 980) dosed twice daily.

FIG. 10 illustrates the cumulative permeation profile of 2.5% CBD gel versus 4.0% CBD gel dosed twice daily.

FIG. 11 illustrates the cumulative permeation profile of 2.5% CBD gel versus 4% CBD (with decreased ethanol and increased propylene glycol) gel through human skin.

FIG. 12 illustrates the cumulative permeation profile of 2.5% CBD gel versus 4% CBD (with either 3.5% or 10% transcutol) gel through human skin.

FIG. 13 is a line graph illustrating the stability rate profiles of CBD in pH 4.0, 5.0, 5.5, 6.0, 6.5 and 7.0.

FIG. 14 is a line graph illustrating the K_{degradation} versus pH profile of cannabidiol in acetate and phosphate buffers at pH 4.0, 5.0, 5.5, 6.0, 6.5, and 7.0.

FIG. 15 illustrates cumulative permeation profile of 2.5% CBD gel with 7.5% transcutol, 2.5% CBD gel with 3.5% transcutol and 4% CBD with 3.5% transcutol through human skin.

FIG. 16 illustrates the cumulative permeation profile of 2.5% CBD gel with 7.5% transcutol, 2.5% CBD gel with 3.5% transcutol and 4% CBD with 3.5% transcutol through human skin.

FIG. 17 illustrates the cumulative permeation profile of 2.5% CBD gel with 7.5% transcutol and 15% propylene glycol, 2.5% CBD gel with 3.5% transcutol and 19% propylene glycol and 2.5% CBD with 3.5% transcutol and 10% propylene glycol through human skin.

FIG. 18 illustrates the cumulative permeation profile of 2.5% CBD gel with 7.5% transcutol and 15% propylene glycol, 2.5% CBD gel with 3.5% transcutol and 10% propylene glycol and 10% CBD with 70% ethanol through human skin.

FIG. 19 illustrates cumulative permeation profile of 1.0% CBD gel with 3.5% transcutol and 10% propylene glycol containing 54.8% EtOH and 2.5% CBD gel with 3.5% transcutol and 10% propylene glycol containing 54.0% EtOH.

### DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the terms “gel” or “gel-like” can be used interchangeably.

As used herein, “cannabinoid” includes any compound that interacts with a cannabinoid receptor and various cannabinoid mimetics, including, but not limited to certain tetrahydropyran analogs (e.g., delta-9-tetrahydrocannabinol, delta-8-tetrahydrocannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-delta-6-tetrahydrocannabinol-1,1-dimethylheptyl, (+)-(3S,4S)-7-hydroxy-delta-6-tetrahydrocannabinol-1,1-dimethylheptyl, 11-hydroxy-delta-9-tetrahydrocannabinol, and delta-8-tetrahydrocannabinol-11-oic acid)); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone), certain open pyran-ring analogs (e.g., 2-[3-methyl-6-(1-methylethenyl)-2-cyclohexen-1-yl]-5-pentyl-1,3-benzenedi-ol and 4-(1,1-dimethylheptyl)-2,3′-dihydroxy-6′ alpha-(3-hydroxypropyl)-1′,-2′,3′,4′,5′,6′-hexahydrobiphenyl), and their pharmaceutically acceptable salts, solvates, metabolites (e.g., cutaneous metabolites), and metabolic precursors.

As used herein, “cannabidiol” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives.

In one embodiment described herein, the cannabinoid, or mixture of cannabinoids, is obtained from the extract from of a natural source, such as plants from the cannabis genus (e.g., Cannabis sativa, Cannabis indicia and Cannabis ruderalis). In an alternative embodiment, the cannabinoid, or mixture of cannabinoids results from synthetic chemical reactions. The synthesis of cannabidiol can be found in Novak et al., Tetrahedron Letters, 23:253 (1982), which is hereby incorporated by reference.

In a further embodiment the cannabinoid is substantially free from impurities. As used herein, “substantially free of impurities” shall mean that impurities, including any cannabinoid not intended to be administered in a therapeutically effective quantity, are present in an amount by weight of the composition of less than about 10%, less than about 5%, less than about 4%, less than about 3%, less than about 2%, less than about 1%, or less than about 0.1%.

One embodiment described herein includes compositions comprising a cannabinoid, such as cannabidiol. A further embodiment described herein includes compositions comprising cannabidiol and a penetration enhancer. In a further embodiment, the composition comprises (a) cannabidiol present in the amount of about 1% to about 98% (wt/wt), such as about 0.1% to about 20% (wt/wt); a lower alcohol having between 1 and 6 carbon atoms that is present in the amount of about 1% to about 98% (wt/wt), such as about 15% to about 85% (wt/wt); a first penetration enhancer that is present in the amount of about 0.1% to about 20% (wt/wt); and water, which is separately added in an amount sufficient for the composition to total 100%, such as about 1% to about 98% (wt/wt). In an additional embodiment, the presence of the lower alcohol is optional and thus the composition would have 0% (wt/wt) of a lower alcohol. In an additional embodiment, the presence water is optional and thus the composition would have 0% (wt/wt) of water.

Another embodiment includes a method of administering a composition to a mammal containing cannabidiol comprising the steps of: (a) preparing a composition comprising cannabidiol present in the amount of about 1% to about 98% (wt/wt), such as about 0.1% to about 20% (wt/wt); a lower alcohol having between 1 and 6 carbon atoms that is present in the amount of about 1% to about 98% (wt/wt), such as about 15% to about 85% (wt/wt); a first penetration enhancer that is present in the amount of about 0.1% to about 20% (wt/wt); and water, which is separately added in an amount sufficient for the composition to total 100%, such as about 1% to about 98% (wt/wt); and (b) administering the composition to the skin of a mammal.

An additional embodiment includes a method of transdermally delivering cannabidiol to a mammal comprising the steps of: (a) preparing a composition comprising cannabidiol present in the amount of about 1% to about 98% (wt/wt), such as about 0.1% to about 20% (wt/wt); a lower alcohol having between 1 and 6 carbon atoms that is present in the amount of about 1% to about 98% (wt/wt), such as about 15% to about 85% (wt/wt); a first penetration enhancer that is present in the amount of about 0.1% to about 20% (wt/wt); and water, which is separately added in an amount sufficient for the composition to total 100%, such as about 1% to about 98% (wt/wt); and (b) administering the composition to the skin of a mammal.

An additional embodiment includes a method of topically delivering cannabidiol to a mammal comprising the steps of: (a) preparing a composition comprising cannabidiol present in the amount of about 1% to about 98% (wt/wt)), such as about 0.1% to about 20% (wt/wt); a lower alcohol having between 1 and 6 carbon atoms that is present in the amount of about 1% to about 98% (wt/wt)), such as about 15% to about 85% (wt/wt); a first penetration enhancer that is present in the amount of about 0.1% to about 20% (wt/wt); and water, which is separately added in an amount sufficient for the composition to total 100%, such as about 1% to about 98% (wt/wt); and (b) administering the composition to the skin of a mammal.

A further embodiment includes the method of treating a medical condition comprising the steps of: (a) preparing a composition comprising cannabidiol present in the amount of about 1% to about 98% (wt/wt)), such as about 0.1% to about 20% (wt/wt); a lower alcohol having between 1 and 6 carbon atoms that is present in the amount of about 1% to about 98% (wt/wt)), such as about 15% to about 85% (wt/wt); a first penetration enhancer that is present in the amount sufficient for the composition to total 100%, such as about 0.1% to about 20% (wt/wt); and water, which is separately added in an amount of about 1% to about 98% (wt/wt); and (b) administering the composition to the skin of a mammal; and wherein the medical condition is selected from the group consisting of: nausea, vomiting, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid osteoarthritis, synovitis, juvenile rheumatoid arthritis, inhibition of hair growth, pancreatitis and alcoholism.

In all embodiments and examples described herein containing cannabidiol, one or more prodrugs of cannabidiol or other cannabinoid may be included with the cannabidiol or substituted for the cannabidiol.

Cannabidiol may be in any suitable form for administration to a mammal such as in the form of a free base, free acid, salt, hydrate, anhydrate, enantiomer, isomer, tautomer, polymorph, or the like, provided that the free base, salt, hydrate, enantiomer, isomer, tautomer, or polymorph is therapeutically active or undergoes conversion within or outside of the body to a therapeutically active form of cannabidiol.

Embodiments described herein comprise cannabidiol and are suitable for transdermal, oral, buccal, sublingual, injectable, topical, follicular, nasal, ocular, rectal or vaginal administration. The compositions described herein include a vehicle or carrier for the administration of cannabidiol (and/or one or more cannabidiol prodrug) as well as optionally including pharmaceutically acceptable excipients such as solvents, thickening agents, neutralizers, solubilizing agents, wetting agents, penetration enhancers, lubricants, emollients, binders, taste enhancers, antioxidants, disintegrates, substances added to mask or counteract a disagreeable odor, fragrances or tastes, and substances added to improve appearance or texture of the composition.

“Pharmaceutically acceptable salts,” or “salts,” include the salt of the parent molecule, such as cannabidiol or a cannabidiol prodrug, suitable for administration to a mammal and includes those prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids. The following list of pharmaceutically acceptable salts is not meant to be exhaustive but merely illustrative as person of ordinary skill in the art would appreciate that other pharmaceutically acceptable salts of cannabidiol and prodrugs of cannabidiol may be prepared such as those identified in Berge et al., “Pharmaceutical Salts,” Journal of Pharmaceutical Sciences, Vol. 66, No. 1, pp. 1-19 (1977) which is hereby incorporated by references in its entirety.

In one embodiment, acid addition salts are prepared from the free base forms using conventional methodologies involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. The following list of organic and inorganic acids is not meant to be exhaustive but merely illustrative as person of ordinary skill in the art would appreciate that other acids may be used to create pharmaceutically acceptable salts of cannabidiol and prodrugs of cannabidiol. In other embodiments, an acid addition salt is reconverted to the free base by treatment with a suitable base. In still other embodiments, the basic salts are alkali metal salts, e.g., sodium salt.

Pharmaceutical Excipients

The pharmaceutical compositions described herein can, if desired, include one or more pharmaceutically acceptable excipients. The term “excipient” herein means any substance, not itself a therapeutic agent, which may be used as a carrier or vehicle for delivery of a therapeutic agent to a subject or combined with a therapeutic agent (e.g., to create a pharmaceutical composition) to improve its handling or storage properties or to permit or facilitate formation of a dose unit of the composition. Excipients include, by way of illustration and not limitation, binders, disintegrants, taste enhancers, solvents, thickening or gelling agents (and any neutralizing agents, if necessary), penetration enhancers, solubilizing agents wetting agents, antioxidants, lubricants, emollients, substances added to mask or counteract a disagreeable odor, fragrances or taste, and substances added to improve appearance or texture of the composition. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of the cannabidiol or cannabidiol prodrug. Suitable excipients can be found, for example, in the “Handbook of Pharmaceutical Excipients”, 6^{th} edition (Rowe, Shesky and Quinn, editors) and in “Remington: The Science and Practice of Pharmacy, 21^{st} ed., both of which are hereby incorporated by reference in their entirety.

In one embodiment, the cannabidiol can be combined with one or more penetration enhancing agent for transdermal or topical delivery. A penetration enhancer is an excipient that aids in the delivery of an active agent into and/or through the stratum corneum. Penetration enhancers are also known as accelerants, adjuvants or sorption promoters. A suitable penetration enhancer for use in the compositions and methods described herein would preferably exhibit one or more of the following qualities: (i) highly potent, with a specific mechanism of action; (ii) exhibit a rapid onset upon administration; (iii) have a predictable duration of action; (iv) have only non-permanent or reversible effects on the skin; (v) chemically stable; (vi) have no or minimal pharmacological effects; (vii) be physically and chemically compatible with other formulation components; (viii) be odorless; (ix) be colorless; (x) be hypoallergenic; (xi) be non-irritating; (xii) be non-phototoxic; (xiii) be non-comedogenic; (xiv) have a solubility parameter approximating that of the skin (10.5 cal/cm³); (xv) be readily available; (xvi) inexpensive; and (xvii) be able to formulated into pharmaceutical compositions for topical or transdermal delivery of an active pharmaceutical agent.

Several classes of chemical compounds, with various mechanisms of action, can be used as penetration enhancers. Set forth below are non-limiting examples of penetration enhancing agents. Sulfoxides, such as dimethylsulfoxide and decylmethylsulfoxide can be used as penetration enhancing agents. Dimethylsulfoxide enhances penetration in part by increasing lipid fluidity and promoting drug partitioning. In contrast, decylmethylsulfoxide enhances penetration by reacting with proteins in the skin that change the conformation of the proteins, which results in the creation of aqueous channels.

Another class of a penetration enhancers are alkanones, such as N-heptane, N-octane, N-nonane, N-decane, N-undecane, N-dodecane, N-tridecane, N-tetradecane and N-hexadecane. Alkanones are thought to enhance the penetration of an active agent by altering the stratum corneum. A further class of penetration enhancers are alkanol alcohols, such as ethanol, propanol, butanol, 2-butanol, pentanol, 2-pentanol, hexanol, octanol, nonanol, decanol and benzyl alcohol. Lower molecular weight alkanol alcohols, i.e., those with 6 or less carbons, may enhance penetration in part by acting as solubilizing agents, while more hydrophobic alcohols may increase diffusion by extracting lipids from the stratum corneum. A further class of penetration enhancers are fatty alcohols, such as oleyl alcohol, caprylic alcohol, decyl alcohol, lauryl alcohol, 2-lauryl alcohol, myristyl alcohol, cetyl alcohol, stearyl alcohol, oleyl alcohol, linoleyl alcohol and linolenyl alcohol. Polyols, including propylene glycol, polyethylene glycol, ethylene glycol, diethylene glycol, triethylene glycol, dipropylene glycol, glycerol, propanediol, butanediol, pentanediol, hexanetriol, propylene glycol monolaurate and diethylene glycol monomethyl ether (transcutol) can also enhance penetration. Some polyols, such as propylene glycol may function as a penetration enhancer by solvating alpha-kertin and occupying hydrogen bonding sites, thereby reducing the amount of active-tissue binding.

Another class of penetration enhancers are amides, including urea, dimethylacetamide, diethyltoluamide, dimethylormamide, dimethyloctamide, dimethyldecamide and biodegradable cyclic urea (e.g., 1-alkyl-4-imidazolin-2-one). Amides have various mechanisms of enhancing penetration. For example, some amides, such as urea increase the hydration of the stratum corneum, act as a keratolytic and create hydrophilic diffusion channels. In contrast, other amides, such as dimethylacetamide and dimethylormamide, increase the partition to keratin at low concentrations, while increasing lipid fluidity and disrupting lipid packaging at higher concentrations. Another class of penetration enhancing agents are pyrrolidone derivatives, such as 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-methyl-pyrrolidone, N-cyclohexylpyrrolidone, N-dimethylaminopropyl-pyrrolidone, N-cocoalkypyrrolidone and N-tallowalkypyrrolidone, as well as biodegradable pyrrolidone derivatives, including fatty acid esters of N-(2-hydroxyethyl)-2-pyrrolidone. In part, pyrrolidone derivatives enhance penetration through interactions with the keratin in the stratum corneum and lipids in the skin structure. An additional class of penetration enhancers are cyclic amides, including 1-dodecylazacycloheptane-2-one (“Azone”), 1-geranylazacycloheptan-2-one, 1-farnesylazacycloheptan-2-one, 1-geranylgeranylazacycloheptan-2-one, 1-(3,7-dimethyloctyl)-azacycloheptan-2-one, 1-(3,7,11-trimethyldodecyl)azacyclohaptan-2-one, 1-geranylazacyclohexane-2-one, 1-geranylazacyclopentan-2,5-dione and 1-farnesylazacyclopentan-2-one. Cyclic amides, such as Azone, enhance the penetration of active agents in part by affecting the stratum corneum's lipid structure, increasing partitioning and increasing membrane fluidity. Additional classes of penetration enhancers include diethanolamine, triethanolamine and hexamethylenlauramide and its derivatives.

Additional penetration enhancers include linear fatty acids, such as octanoic acid, linoleic acid, valeric acid, heptanoic acid, pelagonic acid, caproic acid, capric acid, lauric acid, myristric acid, stearic acid, oleic acid and caprylic acid. Linear fatty acids enhance penetration in part via selective perturbation of the intercellular lipid bilayers. In addition, some linear fatty acids, such as oleic acid, enhance penetration by decreasing the phase transition temperatures of the lipid, thereby increasing motional freedom or fluidity of the lipids. Branched fatty acids, including isovaleric acid, neopentanoic acid, neoheptanoic acid, neonanoic acid, trimethyl hexaonic acid, neodecanoic acid and isostearic acid, are a further class of penetration enhancers. An additional class of penetration enhancers are aliphatic fatty acid esters, such as ethyl oleate, isopropyl n-butyrate, isopropyl n-hexanoate, isopropyl n-decanoate, isopropyl myristate (“IPM”), isopropyl palmitate and octyldodecyl myristate. Aliphatic fatty acid esters enhance penetration by increasing diffusivity in the stratum corneum and/or the partition coefficient. In addition, certain aliphatic fatty acid esters, such as IPM, enhance penetration by directly acting on the stratum corneum and permeating into the liposome bilayers thereby increasing fluidity. Alkyl fatty acid esters, such as ethyl acetate, butyl acetate, methyl acetate, methylvalerate, methylpropionate, diethyl sebacate, ethyl oleate, butyl stearate and methyl laurate, can act as penetration enhancers. Alkyl fatty acid esters enhance penetration in part by increasing the lipid fluidity.

An additional class of penetration enhancers are anionic surfactants, including sodium laurate, sodium lauryl sulfate and sodium octyl sulfate. Anionic surfactants enhance penetration of active agents by altering the barrier function of the stratum corneum and allowing removal of water-soluble agents that normally act as plasticizers. A further class of penetration enhancers are cationic surfactants, such as cetyltrimethylammonium bromide, tetradecyltrimethylammonium, octyltrimethyl ammonium bromide, benzalkonium chloride, octadecyltrimethylammonium chloride, cetylpyridinium chloride, dodecyltrimethylammonium chloride and hexadecyltrimethylammonium chloride. Cationic surfactants enhance penetration by adsorbing at, and interacting with, interfaces of biological membranes, resulting in skin damage. A further class of penetration enhancers are zwitterionic surfactants, such as hexadecyl trimethyl ammoniopropane sulfonate, oleyl betaine, cocamidopropyl hydroxysultaine and cocamidopropyl betaine. Nonionic surfactants, including Polyxamer (231, 182, 184), Polysorbate (20, 60), Brij (30, 93, 96, 99), Span (20, 40, 60, 80, 85), Tween (20, 40, 60, 80), Myrj (45, 51, 52) and Miglyol 840, are yet another class of penetration enhancing agents. Nonionic surfactants enhance penetration in part by emulsifying the sebum and enhancing the thermodynamic activity coefficient of the active.

Further penetration enhancers are bile salts, such as sodium cholate, sodium salts of taurocholic acid, glycolic acids and desoxycholic acids. Lecithin also has been found have penetration enhancing characteristics. An additional class of penetration enhancers are terpenes, which include hydrocarbons, such as d-limonene, alpha-pinene and beta-carene; alcohols, such as, alpha-terpineol, terpinen-4-ol and carvol; ketones, such ascarvone, pulegone, piperitone and menthone; oxides, such as cyclohexene oxide, limonene oxide, alpha-pinene oxide, cyclopentene oxide and 1,8-cineole; and oils such as ylang ylang, anise, chenopodium and eucalyptus. Terpenes enhance penetration in part by disrupting the intercellular lipid bilayer to increase diffusivity of the active and opening polar pathways within and across the stratum corneum. Organic acids, such as salicylic acid and salicylates (including their methyl, ethyl and propyl glycol derivates), citric acid and succinic acid, are penetration enhancers. Another class of penetration enhancers are cyclodextrins, including 2-hydroxypropyl-beta-cyclodextrin and 2,6-dimethyl-beta-cyclodextrin. Cyclodextrins enhance the permeation of active agents by forming inclusion complexes with lipophilic actives and increasing their solubility in aqueous solutions.

Additional penetrations enhancers include, but are not limited to: alkyl-2-(N,N-disubstituted amino)-alkanoate ester (NexAct®); 2-(n-nonyl)-1,3-dioxolane (SEPA®); di(lower)alkyl esters of diacids (e.g., diisopropyl adipate); monoglyceride fatty acids (e.g., glyceryl monolaurate); tetrahydrofurfuryl alcohol; 2-(2-ethoxyethoxy)ethanol; alkylaryl ethers of polyethylene oxide; polyethylene oxide monomethyl ethers; polyethylene oxide dimethyl ethers; acetoacetic ester; oleoyl macrogolglyceride; caprylocaproyl macrogolylyceride; polyoxyethylene 6 caprylic triglyceride; polyoxyethylene glyceride; PPG-5 ceteth-20; lauroyl macroglyceride oleic acid. Additional penetration enhancers suitable for use can also be found in U.S. patent application Ser. No. 10/032,163, which is incorporated by reference herein.

The penetration enhancing agent(s) is/are present in an amount sufficient to provide the desired level of drug transport through the stratum corneum and epidermis. Illustratively, one or more pharmaceutically acceptable penetration enhancer is present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9% or about 10%, about 11%, about 12%, about 11%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

As a further illustration, one or more pharmaceutically acceptable penetration enhancer is present in a total amount by weight of about 0.1% to about 20%, about 0.1% to about 19%, about 0.1% to about 18%, about 0.1% to about 17%, about 0.1% to about 16%, about 0.1% to about 15%, about 0.1% to about 14%, about 0.1% to about 13%, about 0.1% to about 12%, about 0.1% to about 11%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 1% to about 95%; about 5% to about 95%; about 10% to about 95%; about 15% to about 95%; about 20% to about 95%; about 25% to about 95%; about 30% to about 95%; about 35% to about 95%; about 40% to about 95%; about 45% to about 95%; about 50% to about 95%; about 55% to about 95%; about 60% to about 95%; about 65% to about 95%; about 70% to about 95%; about 75% to about 95%; about 80% to about 95%; about 85% to about 95%; about 90% to about 95%; about 1% to about 90%; about 5% to about 90%; about 10% to about 90%; about 15% to about 90%; about 20% to about 90%; about 25% to about 90%; about 30% to about 90%; about 35% to about 90%; about 40% to about 90%; about 45% to about 90%; about 50% to about 90%; about 55% to about 90%; about 60% to about 90%; about 65% to about 90%; about 70% to about 90%; about 75% to about 90%; about 80% to about 90%; about 85% to about 90%; about 1% to about 85%; about 5% to about 85%; about 10% to about 85%; about 15% to about 85%; about 20% to about 85%; about 25% to about 85%; about 30% to about 85%; about 35% to about 85%; about 40% to about 85%; about 45% to about 85%; about 50% to about 85%; about 55% to about 85%; about 60% to about 85%; about 65% to about 85%; about 70% to about 85%; about 75% to about 85%; about 80% to about 85%; about 1% to about 80%; about 5% to about 80%; about 10% to about 80%; about 15% to about 80%; about 20% to about 80%; about 25% to about 80%; about 30% to about 80%; about 35% to about 80%; about 40% to about 80%; about 45% to about 80%; about 50% to about 80%; about 55% to about 80%; about 60% to about 80%; about 65% to about 80%; about 70% to about 80%; about 75% to about 80%; about 1% to about 75%; about 5% to about 75%; about 10% to about 75%; about 15% to about 75%; about 20% to about 75%; about 25% to about 75%; about 30% to about 75%; about 35% to about 75%; about 40% to about 75%; about 45% to about 75%; about 50% to about 75%; about 55% to about 75%; about 60% to about 75%; about 65% to about 75%; about 70% to about 75%; about 1% to about 70%; about 5% to about 70%; about 10% to about 70%; about 15% to about 70%; about 20% to about 70%; about 25% to about 70%; about 30% to about 70%; about 35% to about 70%; about 40% to about 70%; about 45% to about 70%; about 50% to about 70%; about 55% to about 70%; about 60% to about 70%; about 65% to about 70%; about 1% to about 65%; about 5% to about 65%; about 10% to about 65%; about 15% to about 65%; about 20% to about 65%; about 25% to about 65%; about 30% to about 65%; about 35% to about 65%; about 40% to about 65%; about 45% to about 65%; about 50% to about 65%; about 55% to about 65%; about 60% to about 65%; about 1% to about 60%; about 5% to about 60%; about 10% to about 60%; about 15% to about 60%; about 20% to about 60%; about 25% to about 60%; about 30% to about 60%; about 35% to about 60%; about 40% to about 60%; about 45% to about 60%; about 50% to about 60%; about 55% to about 60%; about 1% to about 55%; about 5% to about 55%; about 10% to about 55%; about 15% to about 55%; about 20% to about 55%; about 25% to about 55%; about 30% to about 55%; about 35% to about 55%; about 40% to about 55%; about 45% to about 55%; about 50% to about 55%; about 1% to about 50%; about 5% to about 50%; about 10% to about 50%; about 15% to about 50%; about 20% to about 50%; about 25% to about 50%; about 30% to about 50%; about 35% to about 50%; about 40% to about 50%; about 45% to about 50%; about 1% to about 45%; about 5% to about 45%; about 10% to about 45%; about 15% to about 45%; about 20% to about 45%; about 25% to about 45%; about 30% to about 45%; about 35% to about 45%; about 40% to about 45%; about 1% to about 40%; about 5% to about 40%; about 10% to about 40%; about 15% to about 40%; about 20% to about 40%; about 25% to about 40%; about 30% to about 40%; about 35% to about 40%; about 1% to about 35%; about 5% to about 35%; about 10% to about 35%; about 15% to about 35%; about 20% to about 35%; about 25% to about 35%; about 30% to about 35%; about 1% to about 30%; about 5% to about 30%; about 10% to about 30%; about 15% to about 30%; about 20% to about 30%; about 25% to about 30%; about 1% to about 25%; about 5% to about 25%; about 10% to about 25%; about 15% to about 25%; about 20% to about 25%; about 1% to about 20%; about 5% to about 20%; about 10% to about 20%; about 15% to about 20%; about 1% to about 15%; about 5% to about 15%; or about 10% to about 15%; about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5% to about 8%; about 6% to about 8%; about 7% to about 8%; about 1% to about 7%; about 2% to about 7%; about 3% to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

In one embodiment, the cannabidiol can be combined with a thickening or gelling agent suitable for use in the compositions and methods described herein to increase the viscosity of the composition. Non-limiting examples of thickening agents (aka gelling agents) which may be used to create the composition or be present in the composition herein include neutralized anionic polymers or neutralized carbomers such as polyacrylic acid (CARBOPOL® by Lubrizol Corporation) (see information at http://www.lubrizol.com/carbopol/default.html, incorporated by reference herein), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3. As used herein, a “neutralized carbomer” is a synthetic, high molecular weight polymer, composed primarily of a neutralized polyacrylic acid. Further, when a base is added to neutralize a carbomer solution, the viscosity of the solution increases. Also suitable are other known polymeric thickening agents such as Pemulen® polymeric emulsifiers, Noveon® polycarbophils, and Klucel®. Additional thickening agents, enhancers and adjuvants may generally be found in Remington's The Science and Practice of Pharmacy as well as the Handbook of Pharmaceutical Excipients, Arthur H. Kibbe ed. 2000. Thickening agents or gelling agents are present in an amount sufficient to provide the desired rheological properties of the composition, which include having a sufficient viscosity for forming a gel or gel-like composition, upon the addition of an optional neutralizing agent, that can be applied to the skin of a mammal.

Illustratively, one or more pharmaceutically acceptable thickening agent or gelling agent is present in a total amount by weight of about 0.1%, about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 11%, about 11.5%, about 12%, about 12.5%, about 13%, about 13.5%, about 14%, about 14.5% or about 15%. As a further illustration, one or more pharmaceutically acceptable thickening or gelling agent is present in a total amount by weight of about 0.1% to about 15%; about 0.1% to about 12.5%; about 0.1% to about 10%; about 0.1% to about 7.5%; about 0.1% to about 5%; about 0.1% to about 2.5%; about 0.1% to about 2% about 0.1% to about 1.5%; about 0.1% to about 1%; about 0.5% to about 5.0%; about 0.5% to about 4%; about 0.5% to about 3%; about 0.5% to about 2%; about 0.5% to about 1%; about 1.0% to about 5.0%; about 1% to about 4%; about 1% to about 3%; or about 1% to about 2%.

In one embodiment a neutralizing agent is optionally used to assist in forming a gel or gel-like composition. Suitable neutralizing agents include sodium hydroxide (e.g., as an aqueous mixture), potassium hydroxide (e.g., as an aqueous mixture), ammonium hydroxide (e.g., as an aqueous mixture), triethanolamine, tromethamine (2-amino 2-hydroxymethyl-1,3 propanediol), aminomethyl propanol (AMP), tetrahydroxypropyl ethylene diamine, diisopropanolamine, Ethomeen C-25 (Armac Industrial Division), Di-2 (ethylhexyl)amine (BASF-Wyandotte Corp., Intermediate Chemicals Division), triamylamine, Jeffamine D-1000 (Jefferson Chemical Co.), b-Dimethylaminopropionitrite (American Cyanamid Co.), Armeen CD (Armac Industrial Division), Alamine 7D (Henkel Corporation), dodecylamine and morpholine. The neutralizing agent is present in an amount sufficient to increase viscosity and form a gel or gel-like composition which is suitable for contact with the skin of a mammal. Illustratively, one or more pharmaceutically acceptable neutralizing agent is present in a total amount by weight of about 0.001%, about 0.0015%, about 0.01%, about 0.015%, about 0.1%, about 0.2%, about 0.3%, about 0.4%, about 0.5%, about 0.6%, about 0.7%, about 0.8%, about 0.9%, about 1.0%, 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%. As a further illustration, one or more pharmaceutically acceptable neutralizing agent is present in a total amount by weight of about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2% and about 0.1% to about 1%.

In one embodiment, a solution of sodium hydroxide is used, such as, e.g., 0.01 N, 0.02 N, 0.025 N, 0.05 N, 0.075 N, 0.1 N sodium hydroxide solution, 0.2 N sodium hydroxide solution, 0.5 N sodium hydroxide solution, 1.0 N sodium hydroxide solution, 1.5 N sodium hydroxide solution, 2.0 N sodium hydroxide solution, 10.0 N sodium hydroxide solution, or any other suitable solution for providing a sufficient amount of the aqueous sodium hydroxide to form the desired gel or gel-like composition. In one embodiment, the composition results from combining a gelling agent with a neutralizing agent such as about 1% to about 10% (wt/wt) 0.025 N sodium hydroxide, while in another embodiment about 0.1% to about 1% (wt/wt) 0.25 N sodium hydroxide is used. Of course, other suitable neutralizing agents can be used as can other concentrations and amounts of aqueous sodium hydroxide so long as there is a sufficient amount of OH⁻ ions to assist in the formation of a gel or gel-like composition.

Compositions described herein optionally comprise one or more pharmaceutically acceptable wetting agents as excipients. Non-limiting examples of surfactants that can be used as wetting agents in compositions of the disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters (e.g., sorbitan monolaurate, sorbitan monooleate, sorbitan monopalmitate and sorbitan monostearate), tyloxapol, and mixtures thereof. Such wetting agents, if present, constitute in total about 0.25% to about 15%, about 0.4% to about 10%, or about 0.5% to about 5%, of the total weight of the composition. Illustratively, one or more pharmaceutically acceptable wetting agents are present in a total amount by weight of about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75% or about 10%.

As used herein, a “solubility agent” is any excipient which is added to a pharmaceutical composition to increase the solubility of a solute.

Compositions described herein optionally comprise one or more pharmaceutically acceptable lubricant, including an anti-adherent and/or a glidant. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; polyethylene glycol (“PEG”) (e.g., Carbowax™ 4000 and Carbowax™ 6000); sodium oleate; sodium lauryl sulfate; and magnesium lauryl sulfate. Such lubricants, if present, constitute about 0.1% to about 10%, about 0.2% to about 8%, or about 0.25% to about 5%, of the total weight of the composition. Illustratively, one or more pharmaceutically acceptable lubricant is present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9% or about 10%.

In another embodiment, the compositions described herein optionally comprise an emollient. Illustrative emollients include mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclomethicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soyabean, octyl palmitate, caprylic/capric triglyceride and glyceryl cocoate. An emollient, if present, is present in the compositions described herein in an amount by weight of the composition of about 1% to about 30%, about 3% to about 25%, or about 5% to about 15%. Illustratively, one or more emollients are present in a total amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, by weight.

In one embodiment, the compositions described herein comprise a first antioxidant. Other embodiments described herein comprise a second antioxidant. Illustrative antioxidants include citric acid, butylated hydroxytoluene (BHT), ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine. An antioxidant, if present, is present in the compositions described herein in the amount of about less than 1% by weight. Illustratively, one or more antioxidants are present in the total amount of about 0.025%, about 0.05%, about 0.075%, about 0.1%, about 0.125%, about 0.15%, about 0.175%, about 0.2%, about 0.225%, about 0.25%, about 0.275%, about 0.3%, 0.325%, about 0.35%, about 0.375%, about 0.4%, about 0.425%, about 0.45%, about 0.475%, about 0.5%, about 0.525%, about 0.55%, about 0.575%, about 0.6%, about 0.625%, about 0.65%, about 0.675%, about 0.7%, about 0.725%, about 0.75%, about 0.775%, about 0.8%, about 0.825%, about 0.85%, about 0.875%, about 0.9%, about 0.925%, about 0.95%, about 0.975%, or about 1%, by weight. As a further illustration one or more antioxidants are present in the total amount by weight of about 0.01% to about 1%; about 0.05% to about 0.5% or about 0.05% to about 0.2%.

In one embodiment, the compositions described herein comprise an antimicrobial preservative. Illustrative anti-microbial preservatives include acids, including but not limited to benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, or thimerosal. The anti-microbial preservative, if present, is present in an amount by weight of the composition of about 0.1% to about 5%, about 0.2% to about 3%, or about 0.3% to about 2%, for example about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

Compositions described herein optionally comprise one or more emulsifying agents. The term “emulsifying agent” refers to an agent capable of lowering surface tension between a non-polar and polar phase and includes compounds defined elsewhere as “self emulsifying” agents. Suitable emulsifying agents can come from any class of pharmaceutically acceptable emulsifying agents including carbohydrates, proteins, high molecular weight alcohols, wetting agents, waxes and finely divided solids. The optional emulsifying agent, if present, is present in a composition in a total amount of about 1% to about 25%, about 1% to about 20%, or about 1% to about 15% by weight of the composition. Illustratively, one or more emulsifying agents are present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

In another embodiment, the composition optionally comprises a water miscible solvent, such as propylene glycol. A suitable water miscible solvent refers to any solvent that is acceptable for use in a pharmaceutical composition and is miscible with water. If present, the water miscible solvent is present in a composition in a total amount of about 1% to about 95%, about 2% to about 75%, about 1% to about 25%; about 1% to about 20%; about 3% to about 50%, about 4% to about 40%, about 5% to about 25%; or about 10% to about 22% by weight of the composition. In a further embodiment, the water miscible solvent is present in a composition in an amount of about 1% to about 99%, by weight of the composition, for example about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99%.

Compositions described herein may optionally comprise one or more alcohols. In a further embodiment, the alcohol is a lower alcohol. As used herein, the term “lower alcohol,” alone or in combination, means a straight-chain or branched-chain alcohol moiety containing one to about six carbon atoms. In one embodiment, the lower alcohol contains one to about four carbon atoms, and in another embodiment the lower alcohol contains two or three carbon atoms. Examples of such alcohol moieties include methanol, ethanol, ethanol USP (i.e., 95% v/v), n-propanol, isopropanol, n-butanol, isobutanol, sec-butanol, and tert-butanol. As used herein, the term “ethanol” refers to C₂H₅OH. It may be used as dehydrated alcohol USP, alcohol USP or in any common form including in combination with various amounts of water. If present, the alcohol is present in an amount sufficient to form a composition which is suitable for contact with a mammal. Illustratively, one or more pharmaceutically acceptable alcohol is optionally present in a total amount by weight of 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98%. As a further illustration, one or more pharmaceutically acceptable alcohol is present in a total amount by weight of about 1% to about 98%; about 10% to about 95%; about 15% to about 95%, about 25% to about 75%; about 35% to about 70%; about 35% to about 65%; about 40% to about 50% or about 45% to about 55%.

In a further embodiment water can be separately added to the composition. The amount of water separately added to a formulation is exclusive of the amount of water independently present in the formulation from any other component (e.g., alcohol, neutralizing agent). Water is optionally present in an amount sufficient to form a composition which is suitable for administration to a mammal. Illustratively, water can be separately added by weight in an amount of 0%; about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% about 91%, about 92%, about 93%, about 94%, about 95%, about 96%, about 97%, or about 98%. As a further illustration, water can be separately added by weight in an amount of 0% to about 85%; about 1% to about 98%; about 10% to about 70%; about 10% to about 40%; about 10% to about 35%; about 20% to about 35%; or about 25% to about 30%.

In a further embodiment, water is separately added to the composition in a quantity or amount sufficient to achieve the desired weight of the composition. In an additional embodiment, water is separately added in a quantity sufficient to obtain 100% weight of the composition.

Compositions described herein may optionally comprise one or more binding agents. Binding agents may be either dry or wet. Dry binding agents may include simple and complex carbohydrates (e.g., sucrose, glucose, fructose, maltose, lactose, maltodextrins, starch, modified starches, mannitol, sorbitol, maltitol, xylitol, and erthritol), cellulose, and cellulosic derivatives (e.g., microcrystalline cellulose, carboxymethyl cellulose, and hydroxyethyl cellulose). Wet binder agents may include polyvinyl pyrrolidone, methycellulose, hydroxypropyl cellulose, hydroxypropyl methylcellulose, carboxymethylcellulose, xanthan gum, carrageenan gum, locust bean gum, alginates, and acacia. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate binding agent and the relative concentration of the binding agent.

In another embodiment, the compositions described herein may contain disintegrants, such as sodium starch glycolate, crosspovidone, crosscarmellose, microcrystalline cellulose and starch. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate disintegrant and the relative concentration of the disintegrant.

In a further embodiment, the compositions disclosed herein may contain lubricants, such as magnesium stearate, stearic acid and its pharmaceutically acceptable salts, talc, vegetable oils, and waxes. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate lubricant and the relative concentration of the lubricant.

Compositions described herein may also optionally comprise one or more taste enhancers, such as sweeteners, including aspartame, acesulfame potassium, sucralose and saccharin or taste masking agents, such as flavorings. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate taste enhancer or taste making agent and the relative concentration of the taste enhancer or taste masking agent.

Therapeutic Uses

In one embodiment, compositions disclosed herein comprise cannabidiol in a total amount by weight of the composition of about 0.1% to about 95%. For example, the amount of cannabidiol by weight of the composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13% about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Illustratively, the compositions disclosed herein may comprise a total amount of cannabidiol by weight of 0.1% to about 20%, about 0.1% to about 19%, about 0.1% to about 18%, about 0.1% to about 17%, about 0.1% to about 16%, about 0.1% to about 15%, about 0.1% to about 14%, about 0.1% to about 13%, about 0.1% to about 12%, about 0.1% to about 11%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5% to about 8%; about 6% to about 8%; about 7% to about 8%; about 1% to about 7%; about 2% to about 7%; about 3% to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

A “pharmacologically effective amount” is the amount of the active pharmaceutical agent in the composition which is sufficient to deliver a therapeutically effective amount of the active agent during the dosing interval in which the composition is administered. It will be understood that a therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age, and also includes any nonhuman animal, particularly a domestic or companion animal, illustratively a cat, dog or a horse as well as laboratory animals such as guinea pigs.

The terms “treat”, “treated”, “treating” and “treatment” are to be broadly understood as referring to any response to, or anticipation of, a medical condition in a mammal, particularly a human, and includes but is not limited to:
preventing the medical condition from occurring in a subject, which may or may not be predisposed to the condition, but has not yet been diagnosed with the condition and, accordingly, the treatment constitutes prophylactic treatment for the medical condition;
inhibiting the medical condition, e.g., arresting, slowing or delaying the onset, development or progression of the medical condition; or
relieving the medical condition, e.g., causing regression of the medical condition or reducing the symptoms of the medical condition.

In one embodiment, a therapeutically effective amount of cannabidiol is administered to treat a medical condition selected from the group consisting of: nausea, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritic, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid arthritis, synovitis, juvenile rheumatoid arthritis, inhibition of hair growth, pancreatitis and alcoholism.

In a further embodiment the cannabidiol gels described herein are suitable for use for the relief of the pain of osteoarthritis of the joints, such as the hands, feet, ankles, wrists, shoulders, back, elbows and knees as well as the acute pain due to minor sprains, strains and contusions.

In one embodiment, the pharmaceutical composition containing cannabidiol is administered once daily to a subject in need thereof. In a further embodiment, the pharmaceutical composition containing cannabidiol or a cannabidiol prodrug is administered twice daily to a subject in need thereof. In a further embodiment, the pharmaceutical composition is administered more than twice daily, such as three, four, five, six, seven or eight times daily.

### Pharmaceutical Dosage Forms

The compositions described herein are used in a “pharmacologically effective amount.”

In one embodiment, the amount of the pharmaceutical composition administered to deliver a therapeutically effective amount of the cannabinoid is about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, about 4 g, about 4.1 g, about 4.2 g, about 4.3 g, about 4.4 g, about 4.5 g, about 4.6 g, about 4.7 g, about 4.8 g, about 4.9 g, about 5 g, about 5.1 g, about 5.2 g, about 5.3 g, about 5.4 g, about 5.5 g, about 5.6 g, about 5.7 g, about 5.8 g, about 5.9 g, about 6 g, about 6.1 g, about 6.2 g, about 6.3 g, about 6.4 g, about 6.5 g, about 6.6 g, about 6.7 g, about 6.8 g, about 6.9 g, about 7 g, about 7.1 g, about 7.2 g, about 7.3 g, about 7.4 g, about 7.5 g, about 7.6 g, about 7.7 g, about 7.8 g, about 7.9 g, about 8 g, about 8.1 g, about 8.2 g, about 8.3 g, about 8.4 g, about 8.5 g, about 8.6 g, about 8.7 g, about 8.8 g, about 8.9 g, about 9 g, about 9.1 g, about 9.2 g, about 9.3 g, about 9.4 g, about 9.5 g, about 9.6 g, about 9.7 g, about 9.8 g, about 9.9 g or about 10 g.

Illustratively, the amount of the pharmaceutical composition administered to deliver a therapeutically effective amount of the cannabinoid is about 1 g to about 10 g, about 1 g to about 6 g, about 1 g to about 2 g, or about 2 g to about 4 g.

In one embodiment, the formulation is a gel, gel-like composition, an ointment, a cream or a patch and comprises cannabidiol, optionally one or more penetration enhancing agents, such as transcutol, isopropyl myristate or propylene glycol; a thickening agent, such as neutralized carbomer; a lower alcohol, such as ethanol or isopropanol; and water. In another embodiment, the formulation is a gel, gel-like composition, an ointment, a cream or a patch, further comprised of an aqueous solution of sodium hydroxide or triethanolamine or an aqueous solution of potassium hydroxide, or a combination thereof, in an amount sufficient, as is known in the art, to assist the gelling agent in increasing the viscosity of the composition and forming a gel or gel-like composition.

In another embodiment, the formulation contains an anionic polymer thickening agent precursor such as a carbomer to be combined with a neutralizer in an amount sufficient to increase the viscosity of the composition and form a gel or gel-like composition in the course of forming the composition.

In another embodiment, the formulation contains an anionic polymer thickening agent precursor such as a carbomer which has been combined with a neutralizer in an amount sufficient to increase the viscosity of the composition and form a gel or gel-like composition with a viscosity greater than 1000 cps as measured by a Brookfield RV DVII+ Viscometer with spindle CPE-52, torque greater than 10%, and the temperature maintained at 25° C.

In yet a further embodiment, the formulation contains an anionic polymer thickening agent precursor such as a carbomer which has been combined with a neutralizer selected from the group consisting of sodium hydroxide, ammonium hydroxide, potassium hydroxide, arginine, aminomethyl propanol, tetrahydroxypropyl ethylenediamine, triethanolamine (“TEA”), tromethamine, PEG-15 cocamine, diisopropanolamine, and triisopropanolamine, or combinations thereof in an amount sufficient to neutralize the anionic polymer thickening agent precursor to increase the viscosity of the composition and form a gel or gel-like composition in the course of forming the composition. Suitable neutralizing agents and their use with selected anionic polymer thickening agent precursors are disclosed in “Neutralizing Carbopol® and Pemulen® Polymers in Aqueous and Hydroalcoholic Systems,” Commercial Brochure TDS-237 (October 1998) by Noveon Inc. of Cleveland, Ohio, incorporated by reference herein.

In yet a further embodiment, the formulation contains an anionic polymer thickening agent precursor such as a carbomer which has been combined with a neutralizer which is an aqueous solution of sodium hydroxide such as 0.01 N, 0.02 N, 0.025 N, 0.05 N, 0.075 N, 0.1 N sodium hydroxide, or 1.5 N sodium hydroxide, or 2.0 N sodium hydroxide or any other convenient strength aqueous solution in an amount sufficient to adequately neutralize the polyacrylic acid and increase the viscosity of the composition and form a gel or gel-like composition. In one embodiment, the composition was prepared using from about 1.0% to about 10.0% 0.025N sodium hydroxide. Accordingly, embodiments employing any percentage from about 1.0% to about 10.0% 0.025 N NaOH may be used, such as, e.g., 1.0%, 2.0%, 3.0%, 4.0%, 5.0%, 6.0%, 7.0%, 8.0%, 9.0% or 10% 0.025 N NaOH.

In an embodiment, the viscosity of a composition described herein is about 1,000 cps to about 100,000 cps. Accordingly, the viscosity of the compositions described and disclosed herein may be any amount from about 1,000 cps to about 100,000 cps, such as, e.g., about 1,000, about 2,000, about 3,000, about 4,000, about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000, about 41,000, about 42,000, about 43,000, about 44,000, about 45,000, about 46,000, about 47,000, about 48,000, about 49,000, about 50,000, about 51,000, about 52,000, about 53,000, about 54,000, about 55,000, about 56,000, about 57,000, about 58,000, about 59,000, about 60,000, about 61,000, about 62,000, about 63,000, about 64,000, about 65,000, about 66,000, about 67,000, about 68,000, about 69,000, about 70,000, about 71,000, about 72,000, about 73,000, about 74,000, about 75,000, about 76,000, about 77,000, about 78,000, about 79,000, about 80,000, about 81,000, about 82,000, about 83,000, about 84,000, about 85,000, about 86,000, about 87,000, about 88,000, about 89,000, about 90,000, about 91,000, about 92,000, about 93,000, about 94,000, about 95,000, about 96,000, about 97,000, about 98,000, about 99,000, about 100,000 cps.

In one embodiment, the pH of the pharmaceutical composition is suitable for administration to a mammal. In a further embodiment, the pH of the pharmaceutical composition is suitable for administration to the skin of a mammal. In additional embodiments, the pH of the pharmaceutical composition is suitable for buccal, sublingual, injection, rectal, vaginal, ocular, nasal or oral administration to a mammal. In one embodiment, the pH of the pharmaceutical composition is about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9 or about 10. Illustratively, the pH of the pharmaceutical composition may be from about 3 to about 10, about 4 to about 8, about 4.5 to about 6.5, or about 5 to about 6.

In one embodiment, a single dosage unit of any formulation comprises a therapeutically effective amount or a therapeutically and/or prophylactically effective amount of cannabidiol.

In one embodiment, compositions described herein are suitable for transdermal administration. In another embodiment, transdermally administrable compositions are adapted for administration in and/or around the abdomen, back, chest, legs, arms, scalp or other suitable skin surface and may include formulations in which the cannabidiol is administered in patches, ointments, creams, suspensions, lotions, pastes, gels, sprays, foams or oils.

In another embodiment, compositions described herein which are transdermally administrable include formulations in which the cannabidiol is placed in a glycol, gel or gel-like formulation.

In one embodiment, compositions described herein are suitable for topical administration. In another embodiment, topical administrable compositions are adapted for administration in and/or around the abdomen, back, chest, legs, arms, scalp or other suitable skin surface and may include formulations in which the cannabidiol is administered in patches, ointments, creams, suspensions, lotions, pastes, gels, sprays, foams or oils.

In one embodiment described herein employs a packet having a polyethylene liner compatible with the components of a cannabidol gel or gel-like composition, as described below. The packet may hold a unit dose or multiple dose.

In another embodiment, the methods and compositions employ a composition that is dispensed from a rigid multi-dose container (for example, with a hand pump) having a larger foil packet, for example, of the composition inside the container. Such larger packets can also comprise a polyethylene liner as above. In one embodiment, the multi-dose container comprises an airless pump that comprises a polyethylene lined foil pouch within a canister with a hand pump inserted. In one embodiment, the pump is primed before use, such as, e.g., by fully depressing the pump three times and discarding the gel. In one embodiment, the pump contains enough product to allow for priming and a set number of precise doses. Each pump depression can deliver any amount of cannabidiol suitable for delivering the desired dose. The pouch size, amount dispensed and the delivery volume per depression are not limited to these embodiments and may be changed or adjusted to meet the needs of the patient population.

In one embodiment, the pharmaceutical composition is a gel or gel-like composition, comprising, by weight: 66% EtOH, 21% H₂O, 6% transcutol, 1% CBD, 0.5% IPM, 1.0% gelling agent (e.g., a carbomer, such as Carbopol 980) and 4.5% NaOH (0.1%).

In one embodiment, the pharmaceutical composition is a gel or gel-like composition, comprising, by weight: 63.27% EtOH, 19.73% H₂O, 6% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 59.8% EtOH, 18.2% H₂O, 6% transcutol, 10% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 66% EtOH, 20.5% H₂O, 2.5% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 63.5% EtOH, 20.5% H₂O, 5% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 61% EtOH, 20.5% H₂O, 7.5% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 43.5% EtOH, 20.5% H₂O, 5% transcutol, 20% PEG 550, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 56% EtOH, 20.5% H₂O, 7.5% transcutol, 5% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 51% EtOH, 20.5% H₂O, 7.5% transcutol, 10% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 46% EtOH, 20.5% H₂O, 7.5% transcutol, 15% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 53.5% EtOH, 20.5% H₂O, 15% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 49.75% EtOH, 20.5% H₂O, 3.75% transcutol, 15% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 54.5% EtOH, 15.0% H₂O, 7.5% transcutol, 15% PG, 5% CBD, 0.5% IPM, 0.1% butylated hydroxytoluene, 1.0% gelling agent (e.g., a carbomer, such as Carbopol 980), and 1.4% NaOH (0.1%).

In one embodiment, the pharmaceutical composition is a gel or gel-like composition, comprising, by weight: 54.5% EtOH, 13.9% H₂O, 7.5% transcutol, 15% PG, 5% CBD, 0.5% IPM, 0.1% butylated hydroxytoluene, 0.1% citric acid, 2.0% gelling agent (e.g. Klucel NF), and 1.4% NaOH (0.1%).

In one embodiment, the pharmaceutical composition is a gel or gel-like composition, comprising, by weight: 54.5% EtOH, 15.71% H₂O, 7.5% transcutol, 15% PG, 5% CBD, 0.5% IPM, 0.1% butylated hydroxytoluene, 0.05% citric acid, 1.5% gelling agent (e.g., a carbomer, such as Carbopol 980), and 0.14% triethanolamine.

In one embodiment, the pharmaceutical composition is a gel or gel-like composition, comprising, by weight: 45% EtOH, 27.96% H₂O, 7.5% transcutol, 15% PG, 2.5% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 43.55% EtOH, 27% H₂O, 7.5% transcutol, 15% PG, 5% CBD, 0.5% IPM, 0.1% butylated hydroxytoluene, 0.05% citric acid, 1.25% gelling agent (e.g., a carbomer, such as Carbopol 980), and 0.14% NaOH (1.0%).

In one embodiment, the pharmaceutical composition is a gel or gel-like composition, comprising, by weight: 45.93% EtOH, 28.53% H₂O, 7.5% transcutol, 15% PG, 1.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 44.07% EtOH, 27.39% H₂O, 7.5% transcutol, 15% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 45% EtOH, 26.21% H₂O, 7.5% transcutol, 15% PG, 4.0% CBD, 0.5% IPM, 0.1% butylated hydroxytoluene, 0.05% citric acid, 1.5% gelling agent (e.g., a carbomer, such as Carbopol 980), and 0.14% triethanolamine.

In one embodiment, the pharmaceutical composition is a gel or gel-like composition, comprising, by weight: 40% EtOH, 26.21% H₂O, 7.5% transcutol, 20% PG, 4.0% CBD, 0.5% IPM, 0.1% butylated hydroxytoluene, 0.05% citric acid, 1.5% gelling agent (e.g., a carbomer, such as Carbopol 980), and 0.14% triethanolamine.

In one embodiment, the pharmaceutical composition is a gel or gel-like composition, comprising, by weight: 40% EtOH, 26.46% H₂O, 7.5% transcutol, 20% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 42% EtOH, 26.46% H₂O, 3.5% transcutol, 22% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 40% EtOH, 26.46% H₂O, 10% transcutol, 17.5% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 45% EtOH, 27.96% H₂O, 3.5% transcutol, 19% PG, 2.5% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 45% EtOH, 26.46% H₂O, 3.5% transcutol, 19% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 51% EtOH, 30.96% H₂O, 3.5% transcutol, 10% PG, 2.5% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 69.88% EtOH, 14.24% H₂O, 10% CBD, 0.47% IPM, 0.86% gelling agent (e.g., a carbomer, such as Carbopol 980), and 4.55% NaOH (0.1%).

In one embodiment, the pharmaceutical composition is a gel or gel-like composition, comprising, by weight: 54.0% EtOH, 28.01% H₂O, 3.5% transcutol, 10% PG, 2.5% CBD, 0.5% IPM, 0.1% butylated hydroxytoluene, 1.25% gelling agent (e.g., a carbomer, such as Carbopol 980), and 0.14% triethanolamine.

In one embodiment, the pharmaceutical composition is a gel or gel-like composition, comprising, by weight: 54.8% EtOH, 28.71% H₂O, 3.5% transcutol, 10% PG, 1.0% CBD, 0.5% IPM, 0.1% butylated hydroxytoluene, 1.25% gelling agent (e.g., a carbomer, such as Carbopol 980), and 0.14% triethanolamine.

In one embodiment, the pharmaceutical composition is free or substantially free of alcohol and could comprise, by weight: cannabidiol in an amount of about 0.1% to about 20% of the composition, a first penetration enhancer in an amount of about 0.1% to about 20% of the composition and water in a quantity sufficient for the composition to total 100%.

In further additional embodiments, the pharmaceutical composition is optionally a gel or gel-like composition, comprising, by weight: 40% to 69.88% EtOH, 13.9% to 30.96% H₂O, 2.5% to 10% transcutol, 10% to 22% PG, 1.0% to 10% CBD and about 0.5% WM. In addition to the foregoing composition components, additional embodiments optionally include about 0.1% butylated hydroxytoluene, 0.86 to 2% gelling agent (e.g., a carbomer, such as Carbopol 980), and 0.14% to 4.55% of a suitable neutralizing agent, if desired for use with the selected gelling agent.

In one embodiment, the compositions described herein are suitable for transdermal and/or topical administration.

In another embodiment, the compositions described herein are suitable for oral administration. In another embodiment, compositions described herein that are orally administrable include formulations in which the cannabidiol is administered in tablets, capsules, suspensions, syrups or liquids. In an additional embodiment, the composition maybe formulated as extended release or long acting tablet or capsule. In a further embodiment, the oral dosage form may be enteric coated using compositions and techniques known to a person of ordinary skill in the art.

In one embodiment, compositions described herein are suitable for buccal administration. In another embodiment, compositions described herein that are bucally administrable may include formulations in which the cannabidiol is administered in lozenges, sprays, gels, pastes, dissolvable tablets or dissolvable strips.

In one embodiment, compositions described herein are suitable for sublingual administration. In another embodiment, compositions described herein that are sublingually administrable may include formulations in which the cannabidiol is administered in lozenges, sprays, gels, pastes, dissolvable tablets or dissolvable strips.

In one embodiment, compositions described herein are suitable for injectable administration. In another embodiment, compositions described herein that are administered via injection may include formulations in which the cannabidiol is administered as an intravenous, intrathecal, subcutaneous or depot injection.

In one embodiment, compositions described herein are suitable for rectal administration. In another embodiment, compositions described herein that are rectally administrable may include formulations in which the cannabidiol is placed in suppositories, ointments, creams, suspensions, solutions, lotions, pastes, gels, sprays, foams or oils.

In one embodiment, compositions described herein are suitable for vaginal administration. In another embodiment, compositions described herein that are vaginally administrable may include formulations in which the cannabidiol is placed in suppositories, ointments, creams, suspensions, solutions, lotions, pastes, gels, sprays, foams or oils.

In one embodiment, compositions described herein are suitable for ocular administration. In another embodiment, compositions described herein that are ocularly administrable may include formulations in which the cannabidiol is placed in ointments, suspensions, solutions, gels or sprays.

In one embodiment, compositions described herein are suitable for nasal administration. In another embodiment, compositions described herein that are nasally administrable may include formulations in which the cannabidiol is placed in ointments, suspensions, solutions, lotions, pastes, gels, sprays or mists.

In one embodiment, a pharmaceutical dosage form is prepared as follows: (1) ethanol is placed into a mixing vessel; (2) cannabidiol is added until dissolved; (3) antioxidants are added until dissolved; (4) propylene glycol is added; (5) transcutol is added; (6) isopropyl myristate is added; (7) thickening agent is added; (8) water is added; (9) a neutralizing agent, if needed, is added; and (10) water is added to a quantity sufficient to achieve 100% total weight.

Cannabidiol Prodrugs

The term prodrug as used herein refers to a compound that undergoes a chemical conversion, through a metabolic process or otherwise within the body of the mammal receiving the compound, into its active form which has a pharmacological effect on the mammal. As described herein, cannabidiol prodrugs can be used with or instead of cannabidiol or other cannabinoids.

In one embodiment, illustrative cannabidiol prodrugs include those compounds of Formula (I):

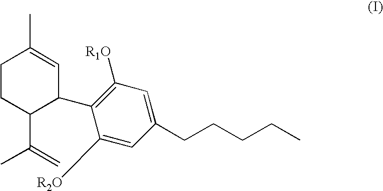

wherein

R₁ and R₂ can be the same or different and are each independently comprised of a hydrogen and/or a bio-labile linker (e.g. ester, oxygenated ester, oxaester, pegylated ester, hydroxylated ester, alkyl ester, amino ester, alkylamino ester, dialkylamino ester, carbonate, alkyl carbonate, carbamate, alkyl carbamate, amino carbamate, alkylamino carbamate, dialkylamino carbamate, or other suitable bio-labile linking structure) and further comprising moieties which can be selected in order to control the rate and extent of absorption and metabolism, including transdermal absorption and metabolism. However, R₁ and R₂ cannot both be a hydrogen atom. Several options for R₁ and R₂ are disclosed herein. Also included herein is the free base, salt, ester, hydrate, amide, enantiomer, isomer, tautomer, polymorph, or derivative thereof of compounds of Formula I.

In a further embodiment, the cannabidiol prodrug can be selected from a group comprising:

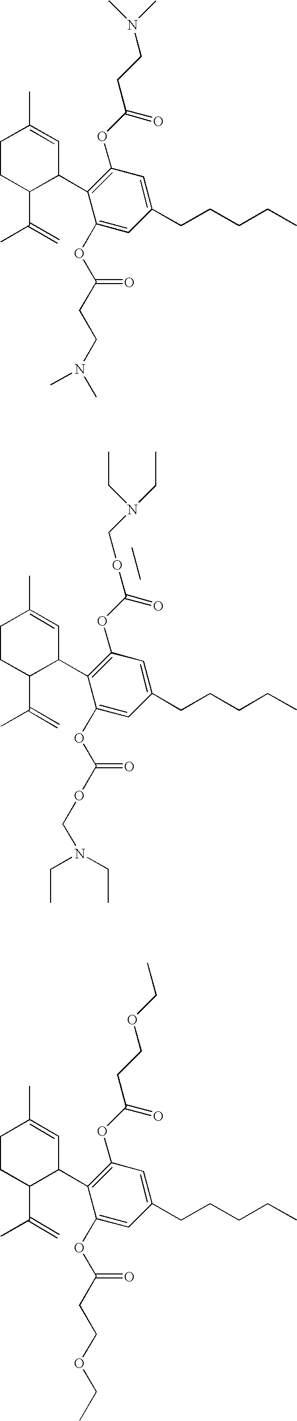

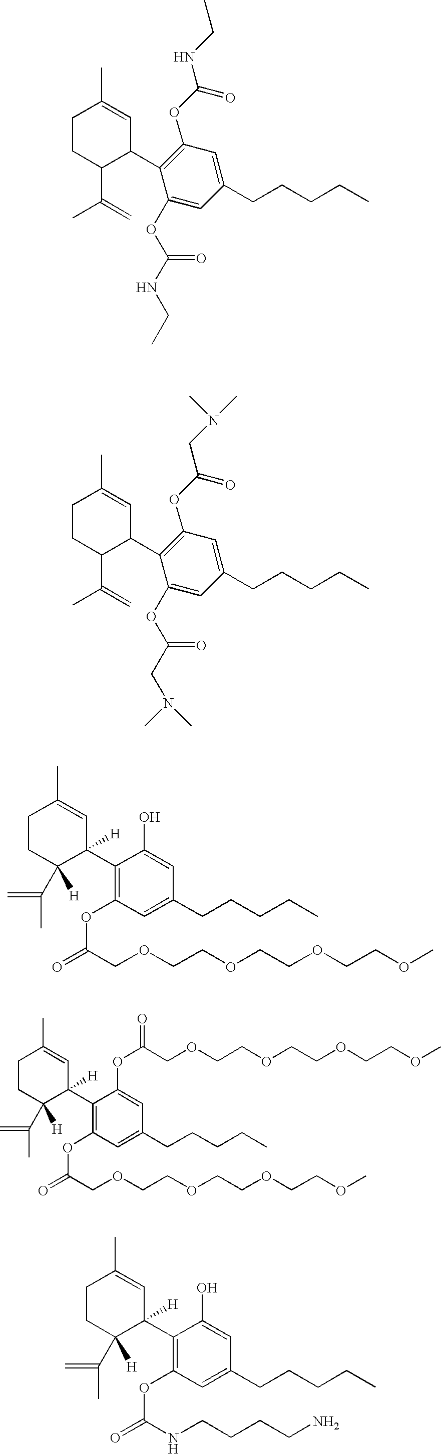

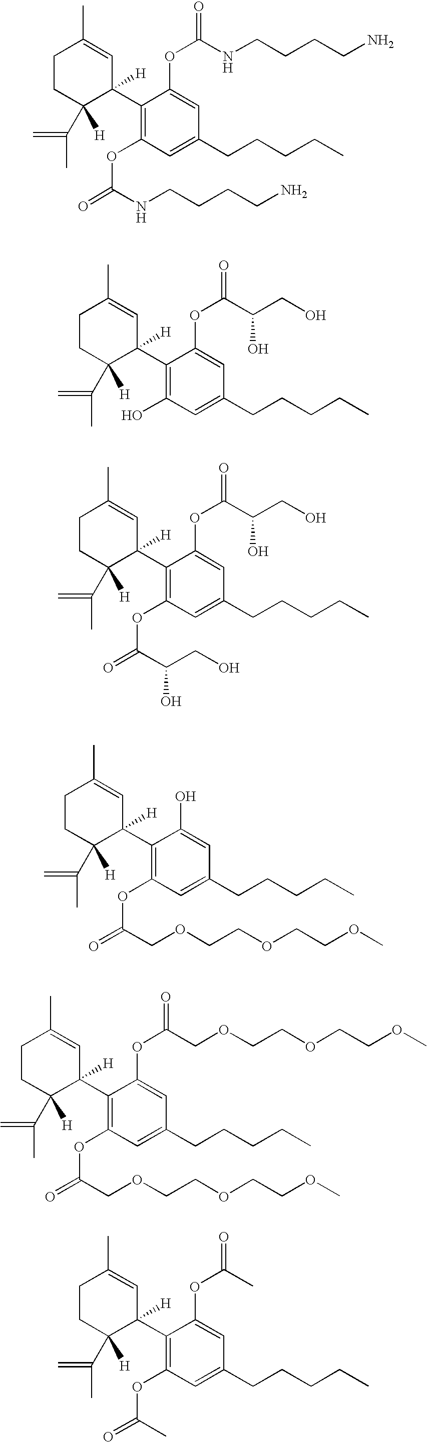

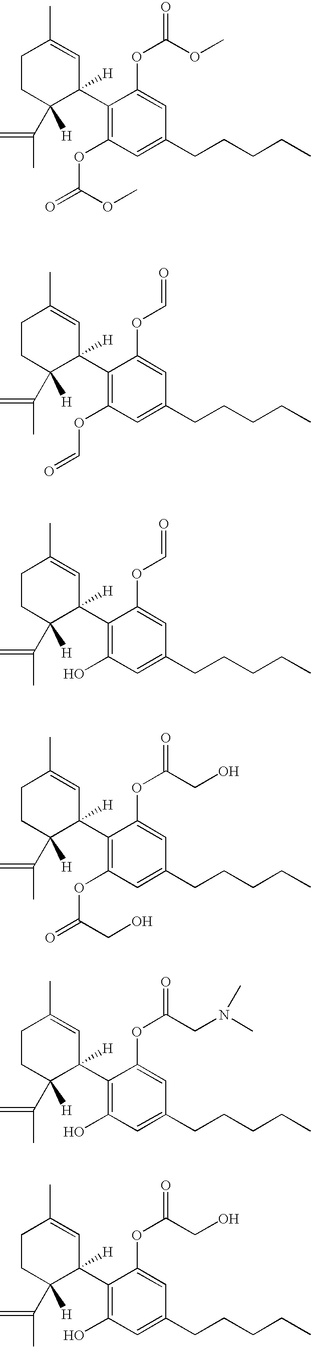

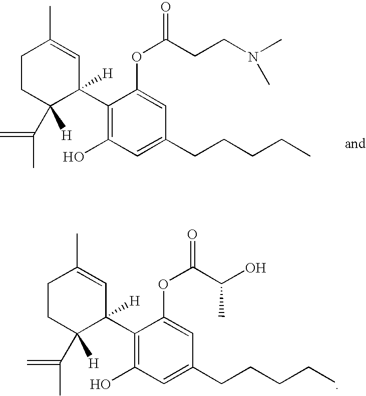

In a further embodiment, one or more cannabidiol prodrug can be used with or instead of cannabidiol or other cannabinoids in the pharmaceutical compositions described herein. In an additional embodiment, a cannabidiol prodrug can be used with or instead of cannabidiol or other cannabinoids in the method of administering cannabidiol to mammal described herein. In an another embodiment, a cannabidiol prodrug can be used with or instead of cannabidiol or other cannabinoids in the method of treating a medical condition by the administration of cannabidiol described herein, wherein the medical condition is selected from a group consisting of nausea, vomiting, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritic, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, juvenile rheumatoid arthritis, inhibition of hair growth, pancreatitis and alcoholism.

Additional embodiments of cannabidiol prodrugs contemplated by the present disclosure include, but are not limited to, those described in U.S. patent application Ser. No. 12/182,974, which is incorporated herein by reference in its entirety.

### EXAMPLES

Several cannabidiol diffusion studies were performed. The tested formulations are set forth in the tables below. The diffusion studies sought to evaluate the permeation through the skin of various concentrations of cannabidiol. Further diffusion studies were performed to evaluate the permeation of CBD by varying the type and amount of the components within the formulation. In addition, pH stability profiles of CBD in buffered solutions were performed to evaluate the pH for long-teem stability. Finally, studies were done to determine the dosing interval necessary to maintain a consistent rate of CBD permeation through human skin samples.

Unless otherwise stated, all diffusion and permeation trials utilized the following experimental method. Human skin harvested during abdominoplasty was used for the diffusion studies. Skin sections were obtained by dermatoming skin, and were stored at −20° C. A PermeGear flow-through (In-Line, Riegelsville, Pa., USA) diffusion cell system was used for the skin permeation studies. The human skin was mounted in 2 cm² diffusion cells. The receiver solution that mimicked circulation was 40% polyethylene glycol (PEG) in water. The diffusion cell was charged with 75 μl of gel and a non-occlusive cap was placed over the cell to allow for gel evaporation. Diffusion cells were kept at 32° C. using a circulating water bath. Studies were run for 24 to 72 hours with either a 12 hour or 24 hour dosing interval. Data was collected from 3-4 cells for the analysis. The samples were diluted 50:50 in acetonitrile (“ACN”) and analyzed by HPLC analysis (see Table A). A cumulative permeation profile was plotted and flux and lag time values were calculated from the linear portion of the plot. Skin disposition was performed by weighing each piece of skin that was in direct contact with drug and extracting the drug from the tissue in 10 mL of ACN to determine concentration per gram of skin.

TABLE A

| | |
|---|---|
| Column | Brownlee ® C₈ reversed phase Spheri 5 μm, |
| | (4.6 × 220 mm) column with a Brownlee ® C₈ reversed |
| | phase 7 μm (3.2 × 150 mm) guard column |
| Mobile phase | 75:25 acetonitrile:0.1% trifluoroacetic acid with 5% |
| | acetonitrile |
| Flow rate | 1.5 mL/min |
| Wavelength | 230 nm |
| Injection | 20 μL |
| volume | |
| Run time | 6 min |
| Retention time | cannabidiol = 5.1 min |

### Example 1

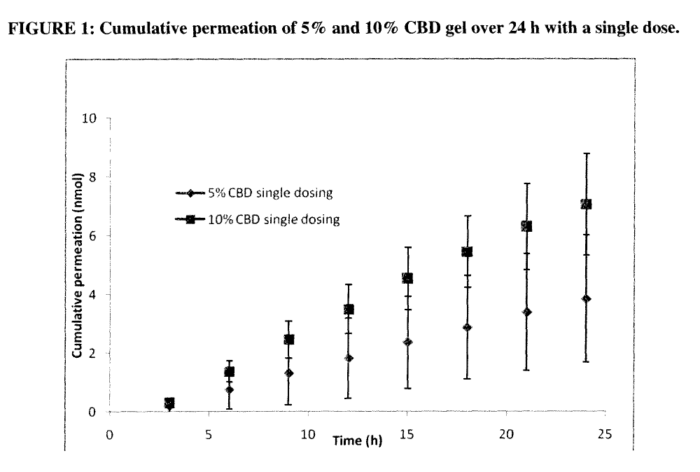

Initial trials were conducted to determine the optimal concentration of CBD. The tested formulations were all gels or gel-like compositions, formed by neutralizing polyacrylic acid (e.g., Carbopol). The compositions of the tested formulations are set forth in Table 1. The results of the permeation and deposition tests are set forth in FIG. 1 and Tables 2 and 3.

TABLE 1

| Comp. | 1% CBD | 5% CBD | 10% CBD | 1% CBD |
|---|---|---|---|---|
| | | | | w/ Oleyl |
| | | | | Alcohol |
| | 66% EtOH | 63.27% EtOH | 59.8% EtOH | 66% EtOH |
| | 21% H₂O | 19.73% H₂O | 18.2% H₂O | 21% H₂O |
| | 6% Transcutol | 6% Transcutol | 6% Transcutol | 6% Oleyl |
| | | | | Alcohol |
| | 1% CBD | 5% CBD | 10% CBD | 1% CBD |
| | 0.5% IPM | 0.5% IPM | 0.5% IPM | 0.5% IPM |
| | 1.0% Carbopol | 1.0% Carbopol | 1.0% Carbopol | 1.0% |
| | 980 | 980 | 980 | Carbopol |
| | | | | 980 |
| | 4.5% NaOH | 4.5% NaOH | 4.5% NaOH | 4.5% NaOH |
| | (0.1%) | (0.1%) | (0.1%) | (0.1%) |
| Total | 100% | 100% | 100% | 100% |

TABLE 2
Permeation data

| | | Lag | Number of |
|---|---|---|---|
| % CBD | Flux (nmol/cm²/h) | time (h) | Samples |
| 1% CBD | 0.0 ± 0.0 | NA | 4 |
| 5% CBD | 0.16 ± 0.07 | 1.93 ± 1.96 | 3 |
| 10% CBD | 0.34 ± 0.07 | 1.89 ± 0.50 | 3 |
| 1% CBD w/ 6% Oleyl | 0.0 ± 0.0 | NA | 4 |

TABLE 3
Skin deposition data and cumulative permeation

| | Drug in skin | Cumulative | |
|---|---|---|---|
| % CBD | (μmol/g) | (nmol) | Number of Samples |
| 1% CBD | 4.9 ± 1.8 | 0.0 ± 0.0 | 4 |
| 5% CBD | 10.1 ± 5.3 | 3.8 ± 2.2 | 3 |
| 10% CBD | 7.5 ± 4.5 | 7.1 ± 1.7 | 3 |
| 1% CBD w 6% Oleyl | 1.99 ± 1.7 | 0.0 ± 0.0 | 4 |

### Example 2

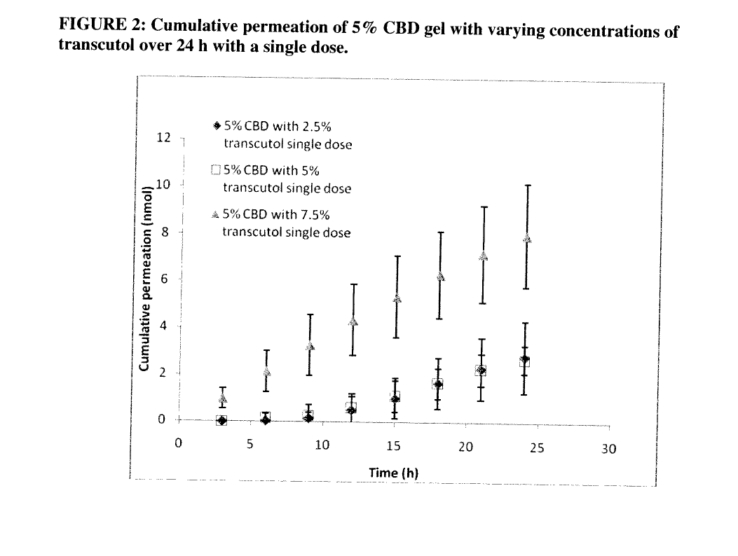

In the next trials, the concentration of transcutol was varied, while the concentration of cannabidiol remained constant. In addition, a formulation with PEG 550 was tested. The compositions of tested formulations are set forth in Table 4. The results of the deposition permeation trials are set forth in FIG. 2 and Tables 5 and 6.

TABLE 4

| Comp. | 5% CBD/ | 5% CBD/ | 5% CBD/ | 5% CBD/ |
|---|---|---|---|---|
| | 2.5% | 5% Transcutol | 7.5% | 5% Transcutol/ |
| | Transcutol | | Transcutol | 20% PEG |
| | 66% EtOH | 63.5% EtOH | 61% EtOH | 43.5% EtOH |
| | 20.5% H₂O | 20.5% H₂O | 20.5% H₂O | 20.5% H₂O |
| | 2.5% | 5% Transcutol | 7.5% | 5% Transcutol |
| | Transcutol | | Transcutol | |
| | 5% CBD | 5% CBD | 5% CBD | 20% PEG 550 |
| | 0.5% IPM | 0.5% IPM | 0.5% IPM | 5% CBD |
| | 1.0% | 1.0% Carbopol | 1.0% Carbopol | 0.5% IPM |
| | Carbopol | 980 | 980 | |
| | 980 | | | |
| | 4.5% NaOH | 4.5% NaOH | 4.5% NaOH | 1.0% Carbopol |
| | (0.1%) | (0.1%) | (0.1%) | 980 |
| | | | | 4.5% NaOH |
| | | | | (0.1%) |
| Total | 100% | 100% | 100% | 100% |

TABLE 5
Permeation data from 5% CBD

| | | | Number of |
|---|---|---|---|
| % Transcutol | Flux (nmol/cm²/h) | Lag time (h) | Samples |
| 2.5% Transcutol | 0.23 ± 0.07 | 9.57 ± 2.05 | 3 |
| 5% Transcutol | 0.18 ± 0.10 | 8.61 ± 3.22 | 4 |
| 7.5% Transcutol | 0.40 ± 0.01 | 2.1 ± 3.0 | 2 |
| 5% Transcutol w/20% | 0.0 ± 0.0 | NA | 4 |
| PEG550 | | | |

TABLE 6
Skin deposition and cumulative permeation data

| | Drug in skin | Cumulative | Number of |
|---|---|---|---|
| % Transcutol | (μmol/g) | (nmol) | Samples |
| 2.5% Transcutol | 15.5 ± 6.2 | 2.8 ± 1.5 | 3 |
| 5% Transcutol | 8.2 ± 2.6 | 2.8 ± 0.5 | 4 |
| 7.5% Transcutol | 6.2 ± 1.8 | 8.0 ± 2.2 | 2 |
| 5% Transcutol w/20% | 3.4 ± 1.9 | 0.0 ± 0.0 | 4 |
| PEG550 | | | |

### Example 3

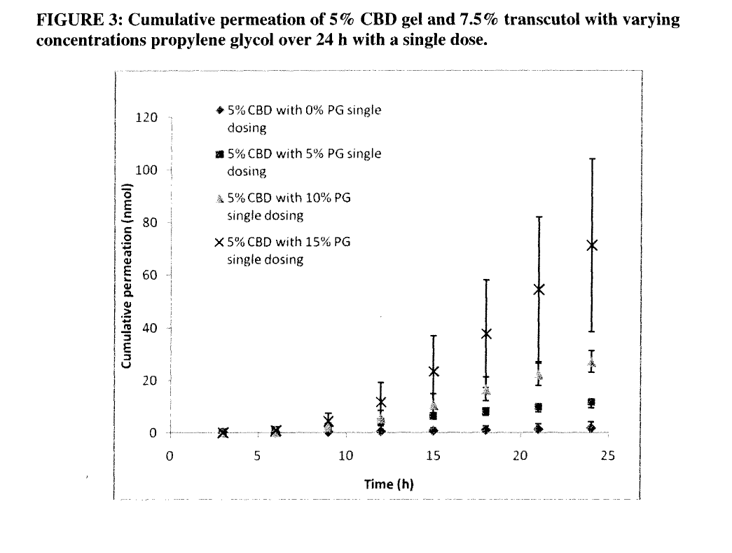

In order to decrease the volatility of the gel formulations without diminishing permeation, different solubilizing agents were examined that would allow for a decrease in the concentration of alcohol. Propylene glycol was chosen as the solubilizer. Comparison studies were then performed by altering the concentration of propylene glycol while maintaining constant concentrations of cannabidiol (5%) and transcutol (7.5%). The formulations tested are set forth in Table 7. The results of the permeation and deposition trials are set forth in FIG. 3 and Tables 8 and 9.

TABLE 7

| Comp. | 0% PG | 5% PG | 10% PG | 15% PG |
|---|---|---|---|---|
| | 61% EtOH | 56% EtOH | 51% EtOH | 46% EtOH |
| | 20.5% H₂O | 20.5% H₂O | 20.5% H₂O | 20.5% H₂O |
| | 7.5% Transcutol | 7.5% Transcutol | 7.5% Transcutol | 7.5% Transcutol |
| | 0% PG | 5% PG | 10% PG | 15% PG |
| | 5% CBD | 5% CBD | 5% CBD | 5% CBD |
| | 0.5% IPM | 0.5% IPM | 0.5% IPM | 0.5% IPM |
| | 1.0% Carbopol 980 | 1.0% Carbopol 980 | 1.0% Carbopol 980 | 1.0% Carbopol 980 |
| | 4.5% NaOH (0.1%) | 4.5% NaOH (0.1%) | 4.5% NaOH (0.1%) | 4.5% NaOH (0.1%) |
| Total | 100% | 100% | 100% | 100% |

TABLE 8
Permeation data

| % Propylene | Flux | Lag time | Number of |
|---|---|---|---|
| Glycol | (nmol/cm²/h) | (h) | Samples |
| 0% PG | 0.03 ± 0.06 | 12.6 ± 2.1 | 3 |
| 5% PG | 0.30 ± 0.06 | 4.4 ± 1.2 | 4 |
| 10% PG | 0.82 ± 0.12 | 5.7 ± 5.4 | 3 |
| 15% PG | 2.81 ± 1.1 | 11.7 ± 1.7 | 4 |

TABLE 9
Skin deposition data

| | Drug in skin | Cumulative | Number of |
|---|---|---|---|
| % PG | (μmol/g) | (nmol) | Samples |
| 0% PG | 7.0 ± 6.1 | 1.4 ± 1.2 | 3 |
| 5% PG | 5.8 ± 2.5 | 11.6 ± 2.2 | 4 |
| 10% PG | 3.5 ± 0.9 | 30.2 ± 5.8 | 3 |
| 15% PG | 5.4 ± 3.0 | 71.4 ± 32.8 | 4 |

### Example 4

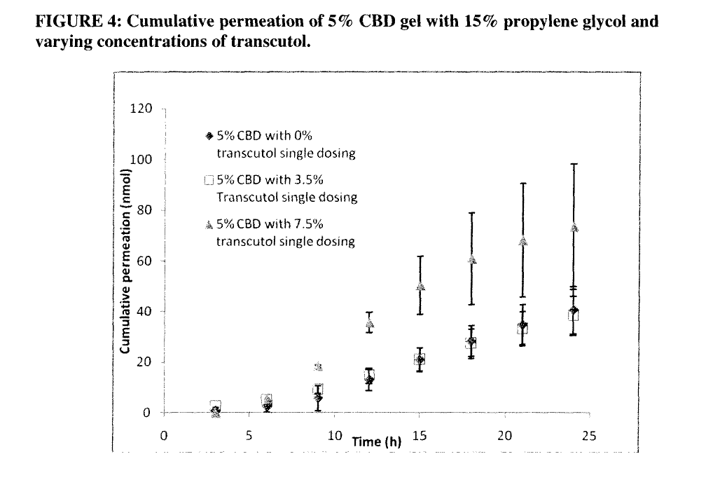

The following example tested formulations in which the concentration of propylene glycol and CBD remained constant, while the concentration of transcutol was varied. The tested formulations are set forth in Table 10. The permeation and disposition results are set forth in FIG. 4 and Tables 11 and 12.

TABLE 10

| Comp. | 0% Transcutol | 3.75% Transcutol | 7.5% Transcutol |
|---|---|---|---|
| | 53.5% EtOH | 49.75% EtOH | 46% EtOH |
| | 20.5% H₂O | 20.5% H₂O | 20.5% H₂O |
| | 0% Transcutol | 3.75% Transcutol | 7.5% Transcutol |
| | 15% PG | 15% PG | 15% PG |
| | 5% CBD | 5% CBD | 5% CBD |
| | 0.5% IPM | 0.5% IPM | 0.5% IPM |
| | 1.0% Carbopol 980 | 1.0% Carbopol 980 | 1.0% Carbopol 980 |
| | 4.5% NaOH (0.1%) | 4.5% NaOH (0.1%) | 4.5% NaOH (0.1%) |
| Total | 100% | 100% | 100% |

TABLE 11
Skin Permeation study results

| | Flux | Lag time | Number of |
|---|---|---|---|
| % Transcutol | (nmol/cm²/h) | (h) | Samples |
| 0% Transcutol | 1.02 ± 0.32 | 3.9 ± 3.3 | 3 |
| 3.75% Transcutol | 0.92 ± 0.16 | 3.2 ± 0.5 | 2 |
| 7.5% Transcutol | 2.31 ± 0.98 | 5.1 ± 0.9 | 3 |

TABLE 12
Skin disposition data and cumulative permeation results

| | Drug in skin | Cumulative | Number of |
|---|---|---|---|
| % Transcutol | (μmol/g) | (nmol) | Samples |
| 0% Transcutol | 1.9 ± 0.3 | 40.5 ± 9.4 | 3 |
| 3.75% Transcutol | 3.7 ± 1.6 | 38.3 ± 7.7 | 2 |
| 7.5% Transcutol | 3.2 ± 1.9 | 62.4 ± 25.8 | 3 |

### Example 5

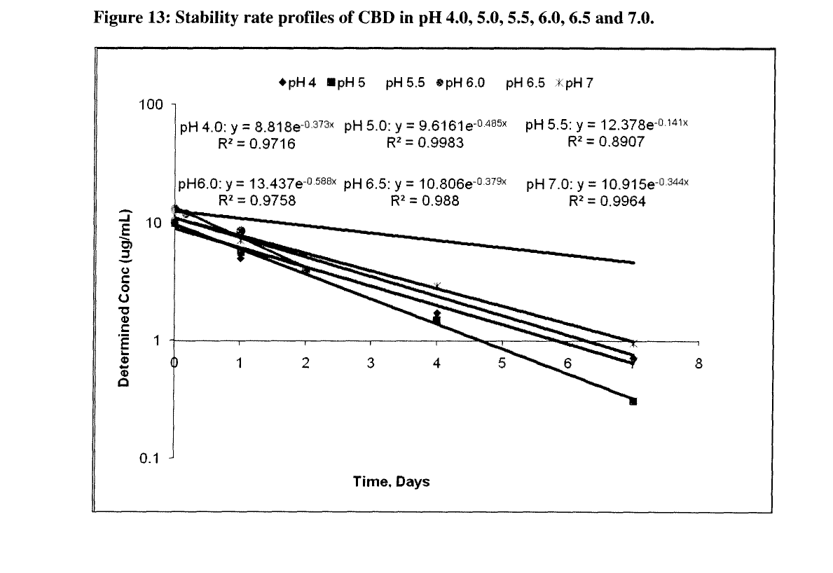

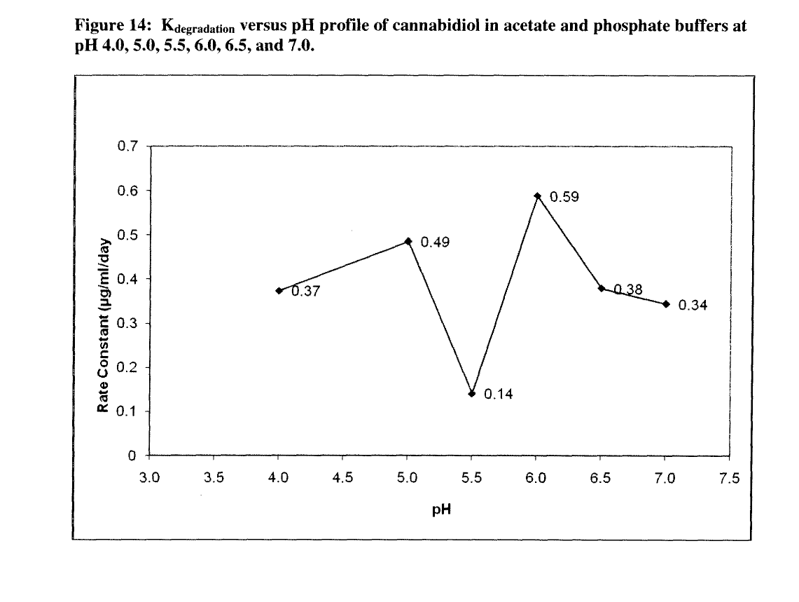

In order to assess the stability of cannabidiol formulations, the pH rate profiles over a range of probable pHs were studied. The rate profile with the lowest K_{deg} would be desirable in order to ensure a two-year shelf life for a cannabidiol gel. The pH ranges studied were 4.0, 5.0, 5.5, 6.0, 6.5 and 7.0 prepared with either an acetate (0.01M: pH 4.0, 5.0 and 5.5) or phosphate (0.01 M: pH 6.0, 6.5 and 7.0) buffer system and maintained at an ionic strength of 0.154 M with NaCl at 40° C. CBD was prepared at 1 mg/mL and analyzed initially at a theoretical 10 μg/ml concentration. The results of these studies appear in FIGS. 13 and 14.

Additional stability studies were conducted with CBD synthesized by alternate routes. The stability sample at 65 days in a 40° C. chamber showed a 28.9 min peak that was approximately 0.74% of the total area compared to the CBD peak at 15.4 min. Over sixty-five days, the purity of CBD is still greater than 99%. Over a sixty-five day accelerated stability study at 40° C., CBD has shown no significant degradation; with purity remaining >98%.

### Example 6

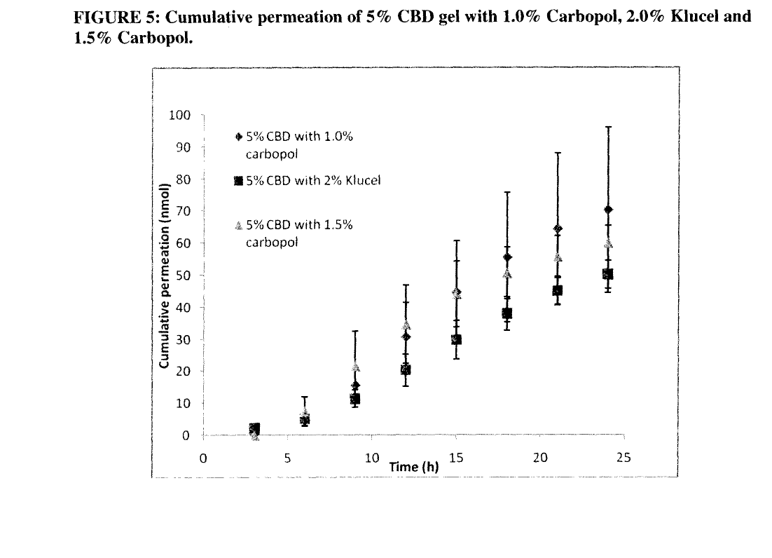

The next trials were conducted to determine the effect of adding antioxidants to the formulation. The formulations included both Carbopol and Klucel gels. The selected antioxidants were butylated hydroxytoluene (“BHT”) and citric acid. In order to solubilize BHT, lower water percentages were utilized. The tested formulations were buffered to a pH of 5.5. The tested formulations are set forth in Table 13. The permeation and disposition results are set forth in FIG. 5 and Tables 14 and 15.

TABLE 13

| Comp. | 5% CBD (Gel 1) | 5% CBD (Gel 2) | 5% CBD (Gel 5) |
|---|---|---|---|
| | 54.5% EtOH | 54.5% EtOH | 54.5% EtOH |
| | 15.0% H₂O | 13.9% H₂O | 15.71% H₂O |
| | 7.5% Transcutol | 7.5% Transcutol | 7.5% Transcutol |
| | 15% PG | 15% PG | 15% PG |
| | 5% CBD | 5% CBD | 5% CBD |
| | 0.5% IPM | 0.5% IPM | 0.5% IPM |
| | 0.1% BHT | 0.1% BHT | 0.1% BHT |
| | 1.0% Carbopol 980 | 0.1% Citric Acid | 0.05% Citric Acid |
| | 1.4% NaOH (0.1%) | 2.0% Klucel NF | 1.5% Carbopol 980 |
| | | 1.4% NaOH (0.1%) | 0.14% |
| | | | Triethanolamine |
| Total | 100% | 100% | 100% |

TABLE 14
Skin Permeation study results

| Formulation | Flux (nmol/cm²/h) | Lag time (h) | Number of Samples |
|---|---|---|---|
| Gel 1 | 2.4 ± 1.1 | 5.1 ± 2.5 | 4 |
| Gel 2 | 1.5 ± 0.3 | 5.3 ± 0.5 | 3 |
| Gel 5 | 2.4 ± 0.5 | 4.5 ± 0.6 | 3 |

TABLE 15
Skin disposition data and cumulative permeation results

| | Drug in skin | Cumulative | Number of |
|---|---|---|---|
| % Transcutol | (μmol/g) | (nmol) | Samples |
| Gel 1 | 2.6 ± 0.4 | 70.3 ± 25.8 | 4 |
| Gel 2 | 3.5 ± 1.3 | 50.2 ± 4.4 | 3 |
| Gel 5 | 4.7 ± 1.7 | 63.9 ± 7.8 | 3 |

### Example 7

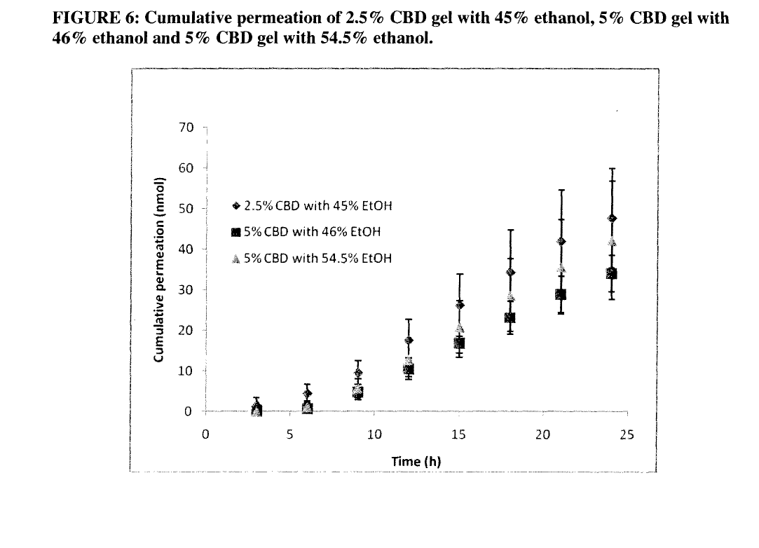

The following example examined the effect of altering the ratio of ethanol to water on permeation. In this example, a 2.5% CBD gel and a 5.0% CBD gel, each with a reduced ratio of ethanol to water, were compared to Gel 5 from the prior Example 6. The tested formulations were buffered to a pH of 5.5. The tested formulations are set forth in Table 16. The permeation and deposition results are set forth in FIG. 6 and Tables 17 and 18.

TABLE 16

| Comp. | 2.5% CBD | 5% CBD | 5% CBD (Gel 5) |
|---|---|---|---|
| | 45% EtOH | 43.55% EtOH | 54.5% EtOH |
| | 27.96% H₂O | 27% H₂O | 15.71% H₂O |
| | 7.5% Transcutol | 7.5% Transcutol | 7.5% Transcutol |
| | 15% PG | 15% PG | 15% PG |
| | 2.5% CBD | 5% CBD | 5% CBD |
| | 0.5% IPM | 0.5% IPM | 0.5% IPM |
| | 0.1% BHT | 0.1% BHT | 0.1% BHT |
| | 0.05% Citric Acid | 0.05% Citric Acid | 0.05% Citric Acid |
| | 1.25% Carbopol 980 | 1.25% Carbopol 980 | 1.5% Carbopol 980 |
| | 0.14% Triethanolamine | 0.14% NaOH (1.0%) | 0.14% NaOH (1.0%) |
| Total | 100% | 100% | 100% |

TABLE 17
Skin Permeation study results

| | Flux | Lag time | Number of |
|---|---|---|---|
| % CBD | (nmol/cm²/h) | (h) | Samples |
| 2.5% low EtOH % | 1.4 ± 0.5 | 5.7 ± 1.1 | 4 |
| 5% low EtOH %* | 0.95 ± 0.1 | 6.7 ± 0.6 | 4 |
| 5% Gel 5 | 1.3 ± 0.4 | 6.8 ± 0.7 | 4 |

TABLE 18
Skin disposition data and cumulative permeation results.

| | Drug in skin | Cumulative | Number of |
|---|---|---|---|
| % CBD | (μmol/g) | (nmol) | Samples |
| 2.5% low EtOH % | 4.5 ± 0.7 | 47.7 ± 12.2 | 4 |
| 5% low EtOH %* | 2.4 ± 0.8 | 34.1 ± 4.5 | 4 |
| 5% Gel 5 | 3.6 ± 0.8 | 42.3 ± 14.5 | 4 |

### Example 8

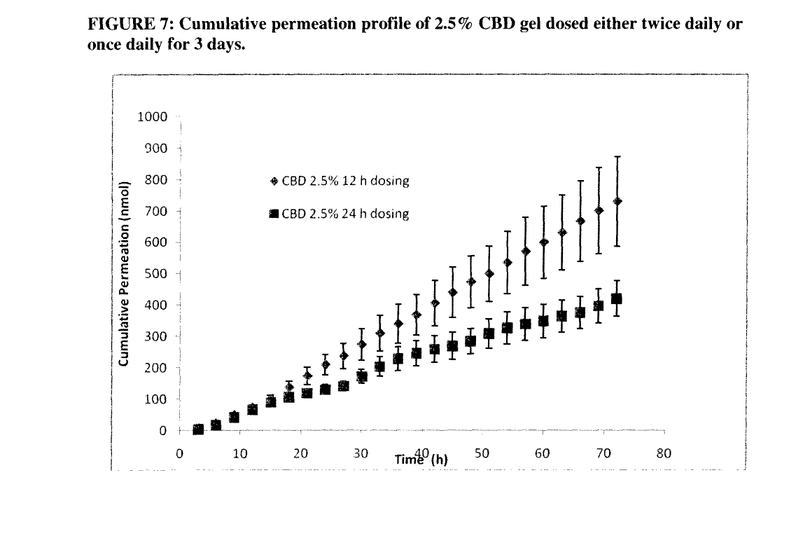

The following example was performed to determine a proper dosing interval. The trial compared a 12-hour versus a 24-hour dosing interval for a formulation comprising 2.5% CBD gel. The two doses were compared over a 72-hour period. In addition, a 1% CBD gel formulation and 4% CBD gel formulation were also tested over a single 24-hour interval. For the purpose of comparison, the ratio of ethanol to water remained constant for all the formulations tested in this example. The tested formulations were buffered to a pH of 5.5. The formulations tested are set forth in Table 19. The permeation and disposition results are set forth in FIG. 7 and Tables 20 and 21.

TABLE 19

| Comp. | 2.5% CBD | 1% CBD | 4% CBD |
|---|---|---|---|
| | 45% EtOH | 45.93% EtOH | 44.07% EtOH |
| | 27.96% H₂O | 28.53% H₂O | 27.39% H₂O |
| | 7.5% Transcutol | 7.5% Transcutol | 7.5% Transcutol |
| | 15% PG | 15% PG | 15% PG |
| | 2.5% CBD | 1.0% CBD | 4.0% CBD |
| | 0.5% IPM | 0.5% IPM | 0.5% IPM |
| | 0.1% BHT | 0.1% BHT | 0.1% BHT |
| | 0.05% Citric Acid | 0.05% Citric Acid | 0.05% Citric Acid |
| | 1.25% Carbopol 980 | 1.25% Carbopol 980 | 1.25% Carbopol 980 |
| | 0.14% Triethanolamine | 0.14% Triethanolamine | 0.14% Triethanolamine |
| Total | 100% | 100% | 100% |

TABLE 20
Skin Permeation study results

| | Flux | Lag time | Number of |
|---|---|---|---|
| % CBD | (nmol/cm²/h) | (h) | Samples |
| 2.5% 12 h dosing/72 h | 5.5 ± 1.9 | 5.7 ± 1.1 | 3 |
| 2.5% 24 h dosing/72 h | 4.6 ± 1.1 | 6.7 ± 0.6 | 4 |
| 1% CBD/24 h | 2.4 ± 1.1 | 6.7 ± 0.6 | 3 |
| 4% CBD/24 h | 5.9 ± 1.6 | 6.8 ± 0.7 | 3 |

TABLE 21
Skin disposition data and cumulative permeation results

| | Drug in skin | Cumulative | Number of |
|---|---|---|---|
| % CBD | (μmol/g) | (nmol) | Samples |
| 2.5% 12 h dosing/72 h | 21.2 ± 10.5 | 729.6 ± 142.7 | 3 |
| 2.5% 24 h dosing/72 h | 12.9 ± 8.0 | 420.7 ± 56.4 | 4 |
| 1% CBD/24 h | 9.8 ± 4.4 | 94.5 ± 41.7 | 3 |
| 4% CBD/24 h | 23.9 ± 12.0 | 160.0 ± 21.9 | 3 |

### Example 9

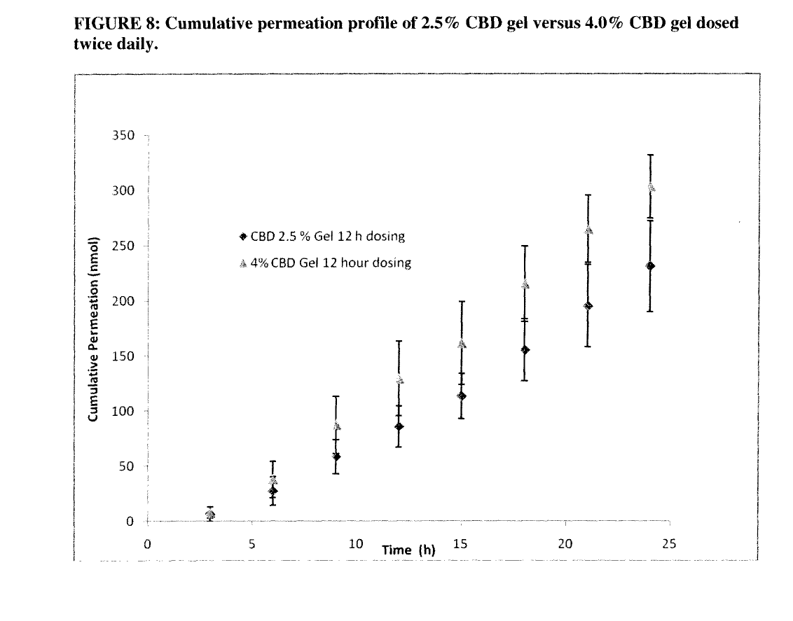

This example was performed to observe any difference in permeation of 2.5% CBD gel versus 4% CBD gel over 24 hours. Both formulations were tested using a 12-hour dosing internal. The tested formulations were buffered to a pH of 5.5. The formulations tested are set forth in Table 22. The permeation and disposition results are set forth in FIG. 8 and Tables 23 and 24.

TABLE 22

| Comp. | 2.5% CBD | 4% CBD |
|---|---|---|
| | 45% EtOH | 44.07% EtOH |
| | 27.96% H₂O | 27.39% H₂O |
| | 7.5% Transcutol | 7.5% Transcutol |
| | 15% PG | 15% PG |
| | 2.5% CBD | 4.0% CBD |
| | 0.5% IPM | 0.5% IPM |
| | 0.1% BHT | 0.1% BHT |
| | 0.05% Citric Acid | 0.05% Citric Acid |
| | 1.25% Carbopol 980 | 1.25% Carbopol 980 |
| | 0.14% Triethanolamine | 0.14% Triethanolamine |
| Total | 100% | 100% |

TABLE 23
Skin Permeation study results

| | | | Number |
|---|---|---|---|
| % CBD | Flux (nmol/cm²/h) | Lag time (h) | of Samples |
| 2.5% 12 h dosing | 5.3 ± 1.3 | 2.0 ± 2.4 | 3 |
| 4.0% 12 h dosing | 6.0 ± 0.9 | 2.2 ± 1.1 | 4 |

TABLE 24
Skin disposition data and cumulative permeation results.

| | Drug in | | Number |
|---|---|---|---|
| % CBD | skin (μmol/g) | Cumulative (nmol) | of Samples |
| 2.5% 12 h dosing | 21.0 ± 2.0 | 263.8 ± 33.4 | 3 |
| 4.0% 12 h dosing | 20.9 ± 14.5 | 286.6 ± 32.6 | 4 |

### Example 10

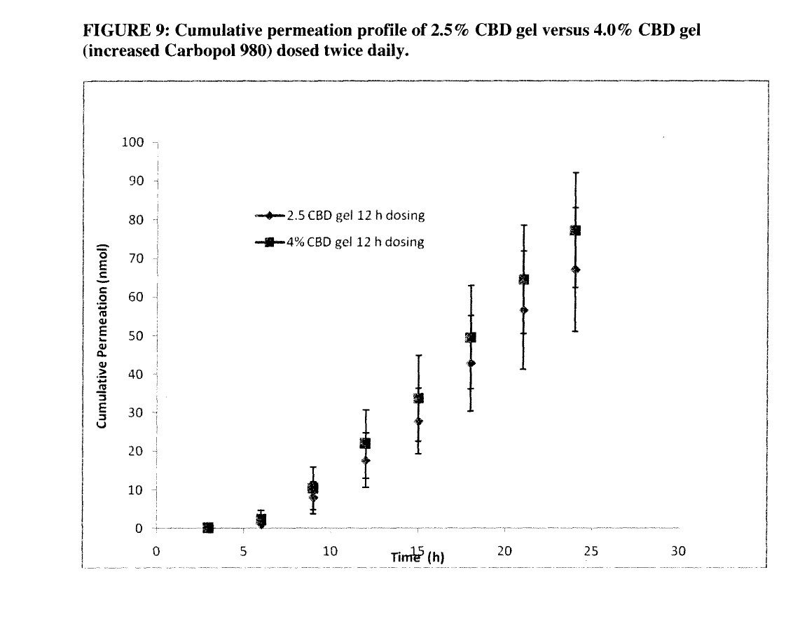

The next example again compared the permeation of a 2.5% CBD gel versus a 4.0% CBD gel over a 24-hour period with a 24-hour dosing interval. However, in this example, a higher percentage of Carbopol 980 was used in the 4.0% CBD formulation to increase the viscosity of the gel. The tested formulations were buffered to a pH of 5.5. The formulations tested are set forth in Table 25. The permeation and disposition results are set forth in FIG. 9 and Tables 26 and 27.

TABLE 25

| Comp. | 2.5% CBD | 4% CBD |
|---|---|---|
| | 45% EtOH | 45% EtOH |
| | 27.96% H₂O | 26.21% H₂O |
| | 7.5% Transcutol | 7.5% Transcutol |
| | 15% PG | 15% PG |
| | 2.5% CBD | 4.0% CBD |
| | 0.5% IPM | 0.5% IPM |
| | 0.1% BHT | 0.1% BHT |
| | 0.05% Citric Acid | 0.05% Citric Acid |
| | 1.25% Carbopol 980 | 1.5% Carbopol 980 |
| | 0.14% Triethanolamine | 0.14% Triethanolamine |
| Total | 100% | 100% |

TABLE 26
Skin Permeation study results

| | | Lag | |
|---|---|---|---|
| % CBD | Flux (nmol/cm²/h) | time (h) | Number of Samples |
| 2.5% 12 h dosing | 2.4 ± 0.2 | 8.4 ± 1.3 | 4 |
| 4% CBD | 2.5 ± 0.2 | 7.5 ± 1.3 | 4 |

TABLE 27
Skin disposition data and cumulative permeation results

| | Drug in skin | Cumulative | Number of |
|---|---|---|---|
| % CBD | (μmol/g) | (nmol) | samples |
| 2.5% 12 h dosing | 1.5 ± 0.3 | 66.9 ± 16.0 | 4 |
| 4.0% 12 h dosing | 2.2 ± 0.5 | 77.2 ± 14.8 | 4 |

### Example 11

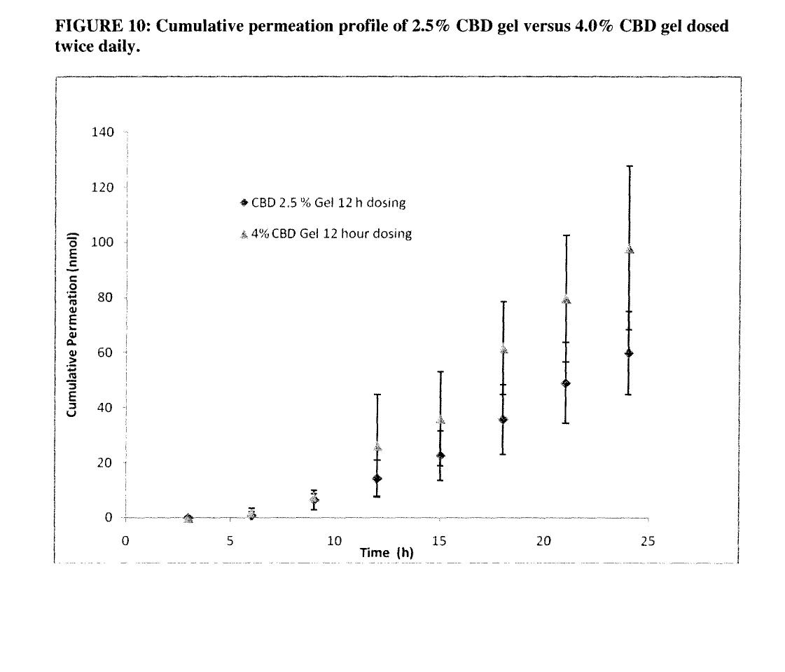

This example again compared the permeation of a 2.5% CBD gel to a 4.0% CBD gel over a 24-hour period using a 12-hour dosing internal. In this example the concentration of ethanol was reduced to 40% and the concentration of propylene glycol was increased to 20%. The formulations used in this example are set forth in Table 28. The tested formulations were buffered to a pH of 5.5. The permeation and disposition results are set forth in FIG. 10 and Tables 29 and 30.

TABLE 28

| Comp. | 2.5% CBD | 4% CBD |
|---|---|---|
| | 45% EtOH | 40% EtOH |
| | 27.96% H₂O | 26.21% H₂O |
| | 7.5% Transcutol | 7.5% Transcutol |
| | 15% PG | 20% PG |
| | 2.5% CBD | 4.0% CBD |
| | 0.5% IPM | 0.5% IPM |
| | 0.1% BHT | 0.1% BHT |
| | 0.05% Citric Acid | 0.05% Citric Acid |
| | 1.25% Carbopol 980 | 1.5% Carbopol 980 |
| | 0.14% Triethanolamine | 0.14% Triethanolamine |
| Total | 100% | 100% |

TABLE 29
Skin Permeation study results

| | | Lag | |
|---|---|---|---|
| % CBD | Flux (nmol/cm²/h) | time (h) | Number of Samples |
| 2.5% 12 h dosing | 1.8 ± 0.2 | 11.7 ± 1.7 | 4 |
| 4.0% 12 h dosing | 4.0 ± 1.6 | 10.0 ± 3.2 | 4 |

TABLE 30
Skin disposition data and cumulative permeation results

| | Drug in skin | Cumulative | Number of |
|---|---|---|---|
| % CBD | (μmol/g) | (nmol) | Samples |
| 2.5% 12 h dosing | 2.2 ± 0.7 | 50.4 ± 10.2 | 4 |
| 4.0% 12 h dosing | 2.1 ± 0.8 | 98.0 ± 29.7 | 4 |

### Example 12

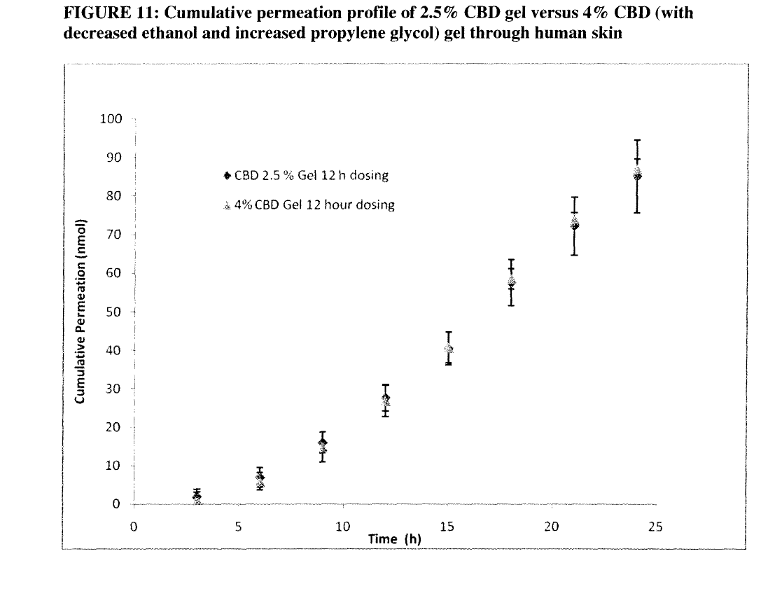

In this example, the permeation of a 2.5% CBD gel was again compared to a 4.0% CBD gel over a 24-hour period, using a 12-hour dosing interval. Although the concentration of Carbopol 980 NF in the 4.0% CBD gel was reduced to 1.25%, the gel remained sufficiently viscous. As in the prior example the concentration of ethanol in the 4.0% CBD gel was 40%, while the propylene glycol concentration of 20%. The tested formulations were buffered to a pH of 5.5. The formulations tested in this example are set forth in Table 31. The permeation and disposition results are set forth in FIG. 11 and Tables 32 and 35.

TABLE 31

| Comp. | 2.5% CBD | 4% CBD |
|---|---|---|
| | 45% EtOH | 40% EtOH |
| | 27.96% H₂O | 26.46% H₂O |
| | 7.5% Transcutol | 7.5% Transcutol |
| | 15% PG | 20% PG |
| | 2.5% CBD | 4.0% CBD |
| | 0.5% IPM | 0.5% IPM |
| | 0.1% BHT | 0.1% BHT |
| | 0.05% Citric Acid | 0.05% Citric Acid |
| | 1.25% Carbopol 980 | 1.25% Carbopol 980 |
| | 0.14% Triethanolamine | 0.14% Triethanolamine |
| Total | 100% | 100% |

TABLE 32
Skin Permeation study results

| | | Lag | |
|---|---|---|---|
| % CBD | Flux (nmol/cm²/h) | time (h) | Number of Samples |
| 2.5% 12 h dosing | 2.7 ± 0.5 | 7.1 ± 1.7 | 4 |
| 4.0% 12 h dosing | 2.8 ± 0.3 | 7.5 ± 1.6 | 3 |

TABLE 33
Skin disposition data and cumulative permeation results

| | Drug in skin | Cumulative | Number of |
|---|---|---|---|
| % CBD | (μmol/g) | (nmol) | Samples |
| 2.5% 12 h dosing | 3.6 ± 0.8 | 85.1 ± 9.5 | 4 |
| 4.0% 12 h dosing | 3.0 ± 0.6 | 87.1 ± 2.5 | 3 |

### Example 13

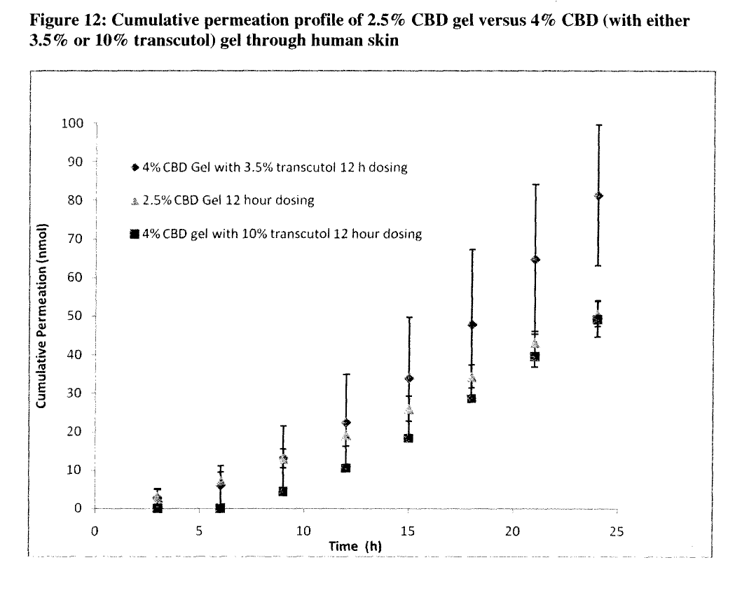

In this example, the permeation of a 2.5% CBD gel was again compared to a 4.0% CBD gel over a 24-hour period, using a 12-hour dosing interval. The concentration of Carbopol 980 NF in the 4.0% CBD gel was maintained at 1.25%. In order to reduce the volatility of the formulation, the concentration of ethanol was fluctuated between 40% and 42%, while the concentration of propylene glycol was correspondingly fluctuated between 17.5% and 22%. The tested formulations were buffered to a pH of 5.5. The concentration of transcutol was adjusted to either 3.5% or 10%. The formulations tested in this example are set forth in Table 34. The permeation and disposition results are set forth in FIG. 12 and Tables 35 and 36.

TABLE 34

| Comp. | 2.5% CBD | 4% CBD | 4% CBD |
|---|---|---|---|
| | | (3.5% transcutol) | (10% transcutol) |
| | 45% EtOH | 42% EtOH | 40% EtOH |
| | 27.96% H₂O | 26.46% H₂O | 26.46% H₂O |
| | 7.5% Transcutol | 3.5% Transcutol | 10% Transcutol |
| | 15% PG | 22% PG | 17.5% PG |
| | 2.5% CBD | 4.0% CBD | 4.0% CBD |
| | 0.5% IPM | 0.5% IPM | 0.5% IPM |
| | 0.1% BHT | 0.1% BHT | 0.1% BHT |
| | 0.05% Citric Acid | 0.05% Citric Acid | 0.05% Citric Acid |
| | 1.25% Carbopol 980 | 1.25% Carbopol 980 | 1.25% Carbopol 980 |
| | 0.14% Triethanolamine | 0.14% Triethanolamine | 0.14% Triethanolamine |
| Total | 100% | 100% | 100% |

TABLE 35
Skin Permeation study results

| % CBD | Flux (nmol/cm²/h) | Lag time (h) | Number of Samples |
|---|---|---|---|
| 2.5% | 1.6 ± 0.1 | 5.9 ± 1.8 | 4 |
| 4.0% (3.5% | 2.9 ± 0.3 | 8.7 ± 2.1 | 3 |
| transcutol) | | | |
| 4.0% (10% | 2.0 ± 0.2 | 9.8 ± 0.8 | 4 |
| transcutol) | | | |

TABLE 36
Skin disposition data and cumulative permeation results.

| | Drug in skin | Cumulative | Number of |
|---|---|---|---|
| % CBD | (μmol/g) | (nmol) | Samples |
| 2.5% | 1.7 ± 0.5 | 50.7 ± 3.3 | 4 |
| 4.0% | 1.6 ± 0.3 | 81.3 ± 18.3 | 3 |
| (3.5% transcutol) | | | |
| 4.0% | 1.4 ± 0.2 | 49.2 ± 4.6 | 4 |
| (10% transcutol) | | | |

### Example 14

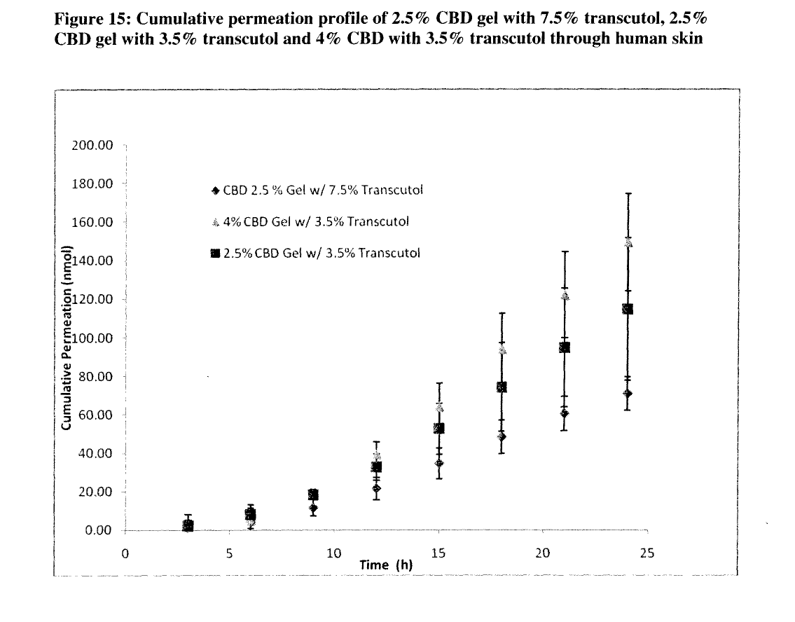

In this example, the permeation of two 2.5% CBD gels, comprising 3.5% and 7.5% w/w of transcutol respectively, were compared to the permeation from a 4% CBD gel with 3.5% w/w of transcutol. The concentration of propylene glycol in the 4% CBD was increased to decrease volatility but maintain solubility of the CBD. The formulations were tested for a twenty-four hour period with a twelve hour dosing interval. The tested formulations were buffered to a pH of 5.5. The formulations tested in this example are set-forth in Table 37. The permeation and disposition results are set forth in FIG. 15 and Tables 38 and 39.

TABLE 37

| Comp. | 2.5% CBD | 2.5% CBD (3.5% transcutol) | 4% CBD |
|---|---|---|---|
| | | | (3.5% transcutol) |
| | 45% EtOH | 45% EtOH | 45% EtOH |
| | 27.96% H₂O | 27.96% H₂O | 26.46% H₂O |
| | 7.5% Transcutol | 3.5% Transcutol | 3.5% Transcutol |
| | 15% PG | 19% PG | 19% PG |
| | 2.5% CBD | 2.5% CBD | 4.0% CBD |
| | 0.5% IPM | 0.5% IPM | 0.5% IPM |
| | 0.1% BHT | 0.1% BHT | 0.1% BHT |
| | 0.05% Citric Acid | 0.05% Citric Acid | 0.05% Citric Acid |
| | 1.25% Carbopol 980 | 1.25% Carbopol 980 | 1.25% Carbopol 980 |
| | 0.14 Triethanolamine | 0.14% Triethanolamine | 0.14% Triethanolamine |
| Total | 100% | 100% | 100% |

TABLE 38
Skin Permeation study results

| | | Lag | |
|---|---|---|---|
| % CBD | Flux (nmol/cm²/h) | time (h) | Number of Samples |
| 2.5% | 2.5 ± 0.4 | 7.6 ± 0.9 | 4 |
| 2.5% | 4.3 ± 1.6 | 6.8 ± 2.1 | 4 |
| (3.5% transcutol) | | | |
| 4.0% | 5.5 ± 1.0 | 8.4 ± 0.2 | 3 |
| (3.5% transcutol) | | | |

TABLE 39
Skin disposition data and cumulative permeation results

| | | Cumulative | |
|---|---|---|---|
| % CBD | Drug in skin (μmol/g) | (nmol) | Number of Samples |
| 2.5% | 4.1 ± 1.9 | 70.8 ± 7.9 | 4 |
| 2.5% (3.5% | 3.6 ± 1.0 | 114.7 ± 31.1 | 4 |
| transcutol) | | | |
| 4.0% (3.5% | 3.1 ± 1.4 | 149.4 ± 25.3 | 3 |
| transcutol) | | | |

### Example 15

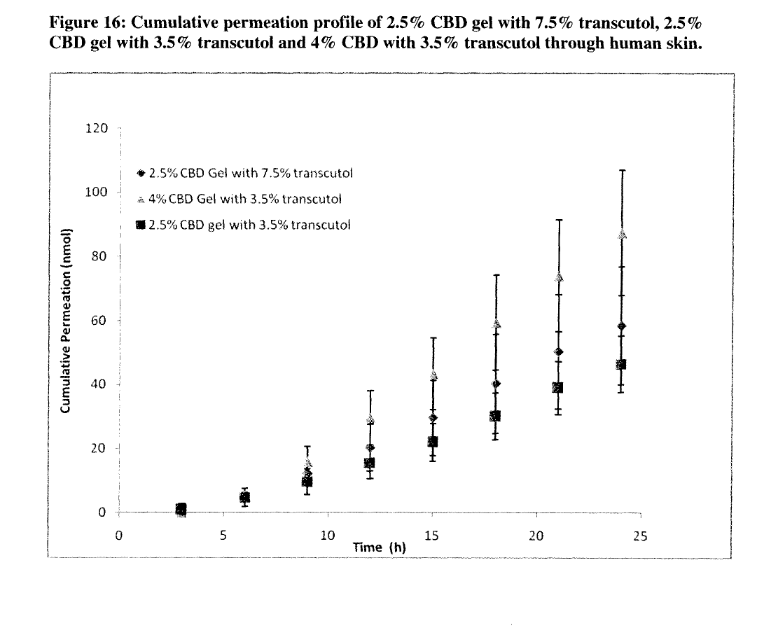

In this example, the permeation of two 2.5% CBD gels, comprising 3.5% and 7.5% w/w of transcutol respectively, were compared to the permeation from a 4% CBD gel with 3.5% w/w of transcutol. The formulations were tested for a twenty-four hour period with a twelve hour dosing interval. The tested formulations were buffered to a pH of 5.5. The formulations tested in this example are set-forth in Table 40. The permeation and disposition results are set forth in FIG. 16 and Tables 41 and 42.

TABLE 40

| Comp. | 2.5% CBD | 2.5% CBD (3.5% transcutol) | 4% CBD |
|---|---|---|---|
| | | | (3.5% transcutol) |
| | 45% EtOH | 45% EtOH | 45% EtOH |
| | 27.96% H₂O | 27.96% H₂O | 26.46% H₂O |
| | 7.5% Transcutol | 3.5% Transcutol | 3.5% Transcutol |
| | 15% PG | 19% PG | 19% PG |
| | 2.5% CBD | 2.5% CBD | 4.0% CBD |
| | 0.5% IPM | 0.5% IPM | 0.5% IPM |
| | 0.1% BHT | 0.1% BHT | 0.1% BHT |
| | 0.05% Citric Acid | 0.05% Citric Acid | 0.05% Citric Acid |
| | 1.25% Carbopol 980 | 1.25% Carbopol 980 | 1.25% Carbopol 980 |
| | 0.14 Triethanolamine | 0.14% Triethanolamine | 0.14% Triethanolamine |
| Total | 100% | 100% | 100% |

TABLE 41
Skin Permeation study results

| % CBD | Flux (nmol/cm²/h) | Lag time (h) | Number of Samples |
|---|---|---|---|
| 2.5% (7.5% | 2.0 ± 0.6 | 6.6 ± 1.6 | 4 |
| transcutol) | | | |
| 2.5% (3.5% | 1.6 ± 0.3 | 7.4 ± 1.2 | 4 |
| transcutol) | | | |
| 4.0% (3.5% | 2.9 ± 0.6 | 6.5 ± 0.7 | 4 |
| transcutol) | | | |

TABLE 42
Skin disposition data and cumulative permeation results

| | Drug | Cumulative | |
|---|---|---|---|
| % CBD | in skin (μmol/g) | (nmol) | Number of Samples |
| 2.5% | 3.4 ± 1.1 | 58.5 ± 18.4 | 4 |
| (7.5% transcutol) | | | |
| 2.5% | 5.5 ± 1.7 | 46.5 ± 8.9 | 4 |
| (3.5% transcutol) | | | |
| 4.0% | 5.8 ± 1.7 | 87.5 ± 19.5 | 4 |
| (3.5% transcutol) | | | |

### Example 16

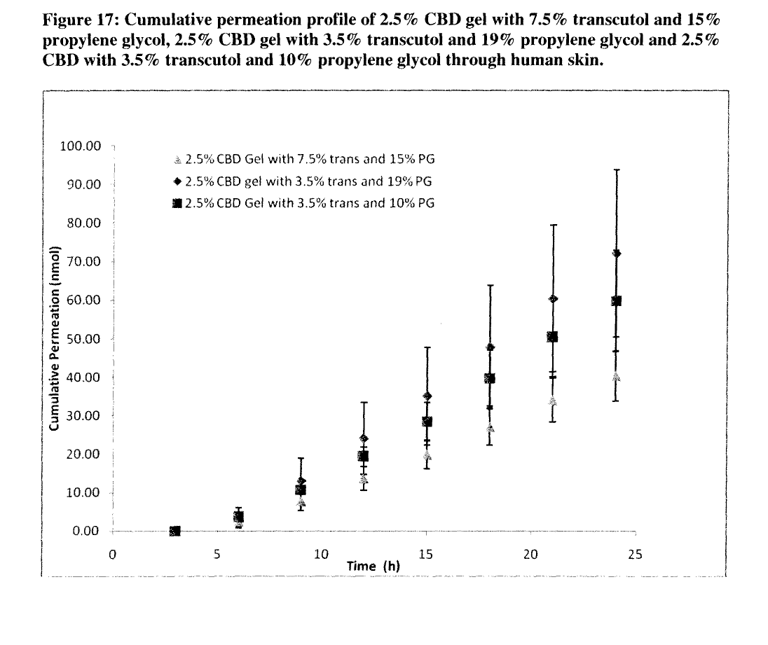

In this example, the permeation of a 2.5% CBD gel with 3.5% transcutol and 19% propylene glycol, a 2.5% CBD gel with 7.5% transcutol and 15% propylene glycol and 2.5% CBD gel with 3.5% transcutol and 10% propylene glycol were compared. The formulations were tested for twenty-four hours with a 12 hour dosing interval. The tested formulations were buffered to a pH of 5.5. The formulations tested in this example are set forth in Table 43. The permeation and disposition results are set forth in FIG. 17 and Tables 44 and 45.

| Comp. | 2.5% CBD | 2.5% CBD (3.5% | 2.5% CBD (3.5% |
|---|---|---|---|
| | | transcutol) | transcutol) |
| | 45% EtOH | 45% EtOH | 51% EtOH |
| | 27.96% H₂O | 27.96% H₂O | 30.96% H₂O |
| | 7.5% Transcutol | 3.5% Transcutol | 3.5% Transcutol |
| | 15% PG | 19% PG | 10% PG |
| | 2.5% CBD | 2.5% CBD | 2.5% CBD |
| | 0.5% IPM | 0.5% IPM | 0.5% IPM |
| | 0.1% BHT | 0.1% BHT | 0.1% BHT |
| | 0.05% Citric Acid | 0.05% Citric Acid | 0.05% Citric Acid |
| | 1.25% Carbopol 980 | 1.25% Carbopol 980 | 1.25% Carbopol 980 |
| | 0.14% Triethanolamine | 0.14% Triethanolamine | 0.14% Triethanolamine |
| Total | 100% | 100% | 100% |

TABLE 44
Skin Permeation study results

| % CBD | | Lag time | |
|---|---|---|---|
| (% PG/% Trans) | Flux (nmol/cm²/h) | (h) | Number of Sample |
| 2.5% (15%/7.5%) | 1.3 ± 0.2 | 6.5 ± 1.0 | 4 |
| 2.5% (19%/3.5%) | 2.4 ± 0.7 | 6.7 ± 1.7 | 4 |
| 2.5% (10%/3.5%) | 2.1 ± 0.5 | 7.0 ± 1.1 | 4 |

TABLE 45
Skin disposition data and cumulative permeation results.

| % CBD | | Cumulative | Number of |
|---|---|---|---|
| (% PG/% Trans) | Drug in skin (μmol/g) | (nmol) | Samples |
| 2.5% (15%/7.5%) | 2.6 ± 0.8 | 40.4 ± 6.5 | 4 |
| 2.5% (19%/3.5%) | 2.9 ± 0.7 | 72.1 ± 21.7 | 4 |
| 2.5% (10%/3.5%) | 4.0 ± 1.4 | 59.8 ± 13.1 | 4 |

### Example 17

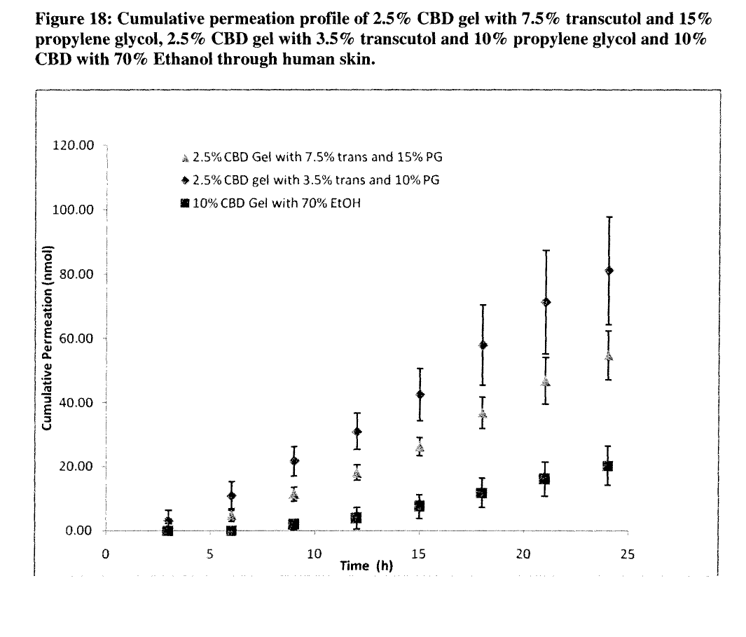

In this example, the permeation of a 2.5% CBD gel with 3.5% transcutol and 10% propylene glycol, a 2.5% CBD gel with 7.5% transcutol and 15% propylene glycol, and a 10% CBD with 70% EtOH and no transcutol and propylene glycol were compared. The formulations were tested for twenty-four hours with a 12 hour dosing interval. The tested formulations were buffered to a pH of 5.5. The formulations tested in this example are set forth in Table 46. The permeation and disposition results are set forth in FIG. 18 and Tables 47 and 48.

TABLE 46

| Comp. | 2.5% CBD | 2.5% CBD (3.5% transcutol) | 10% CBD |
|---|---|---|---|
| | | | (70% EtOH) |
| | 45% EtOH | 51% EtOH | 69.88% EtOH |
| | 27.96% H₂O | 30.96% H₂O | 14.24% H₂O |
| | 7.5% Transcutol | 3.5% Transcutol | 10% CBD |
| | 15% PG | 10% PG | 0.47% IPM |
| | 2.5% CBD | 2.5% CBD | 0.86% Carbopol 980 |
| | 0.5% IPM | 0.5% IPM | 4.55% NaOH |
| | 0.1% BHT | 0.1% BHT | |
| | 0.05% Citric Acid | 0.05% Citric Acid | |
| | 1.25% Carbopol 980 | 1.25% Carbopol 980 | |
| | 0.14% Triethanolamine | 0.14% Triethanolamine | |
| Total | 100% | 100% | 100% |

TABLE 47
Skin Permeation study results

| % CBD | | | Number of |
|---|---|---|---|
| (% PG/% Trans) | Flux (nmol/cm²/h) | Lag time (h) | Samples |
| 2.5% (15%/7.5%) | 1.9 ± 0.5 | 6.9 ± 2.2 | 4 |
| 2.5% (10%/35%) | 2.7 ± 0.8 | 5.9 ± 1.7 | 4 |
| 10% (0%/0%) | 0.8 ± 0.2 | 9.9 ± 2.0 | 3 |

TABLE 48
Skin disposition data and cumulative permeation results

| % CBD | | Cumulative | Number of |
|---|---|---|---|
| (% PG/% Trans) | Drug in skin (μmol/g) | (nmol) | Samples |
| 2.5% (15%/7.5%) | 3.1 ± 1.2 | 54.7 ± 7.6 | 4 |
| 2.5% (10%/3.5%) | 4.3 ± 1.3 | 81.0 ± 16.8 | 4 |
| 10% (0%/0%) | 1.8 ± 0.6 | 20.2 ± 6.1 | 3 |

### Example 18

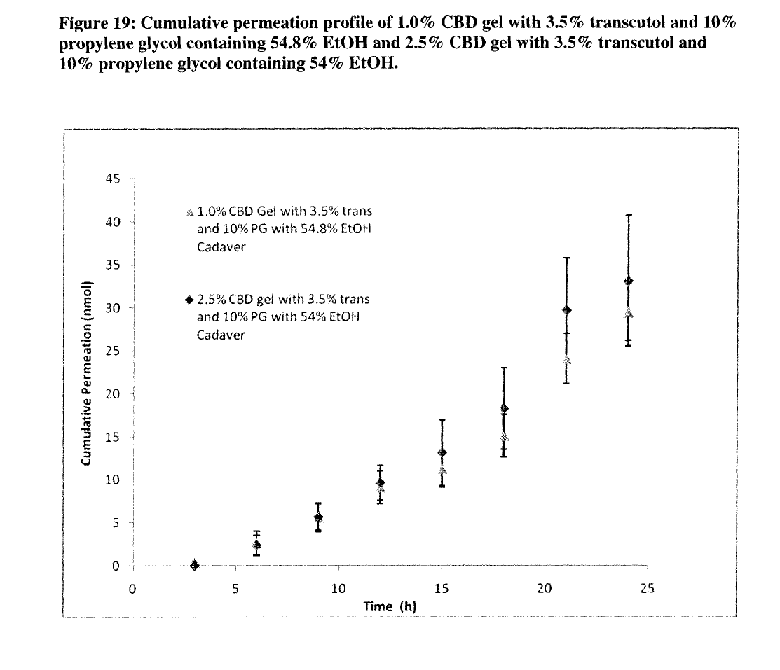

In this example, the permeation of a 1.0% CBD gel with 3.5% transcutol and 10% propylene glycol, a 2.5% CBD gel with 3.5% transcutol and 10% propylene glycol, containing 54.8 and 54.0% EtOH, respectively, were compared. The formulations were tested for twenty-four hours with a 12 hour dosing interval. The tested formulations were buffered to a pH of 5.5. The formulations tested in this example are set forth in Table 49. The permeation and disposition results are set forth in FIG. 19 and Tables 50 and 51.

TABLE 49

| Comp. | 1.0% CBD | 2.5% CBD |
|---|---|---|
| | 54.8% EtOH | 54.0% EtOH |
| | 28.71% H₂O | 28.01% H₂O |
| | 3.5% Transcutol | 3.5% Transcutol |
| | 10% PG | 10% PG |
| | 1.0% CBD | 2.5% CBD |
| | 0.5% IPM | 0.5% IPM |
| | 0.1% BHT | 0.1% BHT |
| | 1.25% Carbopol 980 | 1.25% Carbopol 980 |
| | 0.14% Triethanolamine | 0.14% Triethanolamine |
| Total | 100% | 100% |

TABLE 50
Skin Permeation study results

| % CBD | | | Number of |
|---|---|---|---|
| (% PG/% Trans) | Flux (nmol/cm²/h) | Lag time (h) | Samples |
| 1.0% (10%/3.5%) | 1.0 ± 0.1 | 6.3 ± 1.9 | 6 |
| 2.5% (10%/3.5%) | 1.1 ± 0.3 | 6.8 ± 1.1 | 6 |

TABLE 51
Skin disposition data and cumulative permeation results

| % CBD | Drug in skin | Cumulative | Number of |
|---|---|---|---|
| (% PG/% Trans) | (μmol/g) | (nmol) | Samples |
| 1.0% (10%/3.5%) | 1.7 ± 0.2 | 29.5 ± 3.3 | 6 |
| 2.5% (10%/3.5%) | 1.9 ± 1.0 | 33.1 ± 7.6 | 6 |

Observations

As shown in the Examples above, a 12-hour dosing interval provided a constant linear permeation profile, whereas the 24-hour interval did not. A 2.5% gel produced a greater flux rate than a 1% gel and increasing the concentration to 4% had little or no further significant effect on flux rates. The 2.5% CBD gel 12 h dosing had approximately twice as much drug in skin and total permeation as compared to only a single dose of 2.5% CBD gel per day. Also the 2.5% gel resulted in greater accumulation than the 1% gel, whereas increasing the concentration to 4% did not further increase skin accumulation. Overall, flux values, lag times, CBD skin concentrations, and cumulative permeation were nearly identical suggesting a maximum skin concentration has been achieved, thus additional compound will not significantly enhance permeation. When, however, the ethanol concentration was increased in the 1.0% and 2.5% CBD gel, the performance of the 1.0% CBD gel was essentially the same as the 2.5% CBD gel, suggesting a smaller drug load could be used in the hydroalcoholic gel without a change in effect (Example 18).

All references, including publications, patent applications, and patents, cited herein are hereby incorporated by reference to the same extent as if each reference were individually and specifically indicated to be incorporated by reference and were set forth in its entirety herein.

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

We claim:

1. A pharmaceutical composition comprising:
a. cannabidiol present in an amount of about 0.1% to about 20% (wt/wt) of the composition;
b. a lower alcohol having between 1 and 6 carbon atoms present in an amount of about 15% to about 95% (wt/wt) of the composition;
c. a first penetration enhancer present in an amount of about 0.1% to about 20% (wt/wt) of the composition; and
d. water in a quantity sufficient for the composition to total 100% (wt/wt).

2. The pharmaceutical composition of claim 1, wherein the cannabidiol is present in an amount of about 1% to about 10% (wt/wt) of the composition.

3. The pharmaceutical composition of claim 1, wherein the cannabidiol is present in an amount of about 1% to about 5% (wt/wt) of the composition.

4. The pharmaceutical composition of claim 1, wherein the cannabidiol is present in an amount of about 2.5% (wt/wt) of the composition.

5. The pharmaceutical composition of claim 1, wherein the cannabidiol is present in an amount of about 1% (wt/wt) of the composition.

6. The pharmaceutical composition of claim 1, wherein the first penetration enhancer is selected from the group consisting of: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, diethylene glycol monoethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes.

7. The pharmaceutical composition of claim 1, wherein the first penetration enhancer is present in an amount of about 0.1% to about 15% (wt/wt) of the composition.

8. The pharmaceutical composition of claim 1, wherein the first penetration enhancer is present in an amount about 3% to about 8% (wt/wt) of the composition.

9. The pharmaceutical composition of claim 1, wherein the first penetration enhancer diethylene glycol monoethyl ether and is present in an amount of about 7.5% (wt/wt) of the composition.

10. The pharmaceutical composition of claim 1, wherein the composition further comprises a second penetration enhancer.

11. The pharmaceutical composition of claim 10, wherein the first penetration enhancer comprises diethylene glycol monoethyl ether or oleyl alcohol and the second penetration enhancer comprises isopropyl myristate.

12. The pharmaceutical composition of claim 1, wherein the lower alcohol is ethanol or isopropyl alcohol.

13. The pharmaceutical composition of claim 1, wherein the lower alcohol is present in an amount of about 25% to about 75% (wt/wt) of the composition.

14. The pharmaceutical composition of claim 1, wherein the lower alcohol is present in an amount of about 40% to about 70% (wt/wt) of the composition.

15. The pharmaceutical composition of claim 1, wherein the lower alcohol is ethanol and present in an amount of about 45% to about 55% (wt/wt) of the composition.

16. The pharmaceutical composition of claim 1, further comprising a thickening agent.

17. The pharmaceutical composition of claim 16, wherein the thickening agent is selected from the group consisting of: carboxypolymethylene, carboxymethylcellulose, acrylic acid polymer, neutralized acrylic acid polymer, partially neutralized polyacrylic acid, Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, Pemulen®, Noveon®, polycarbophils, Klucel® and combinations of the foregoing.

18. The pharmaceutical composition of claim 16, wherein the thickening agent is present in an amount of about 0.1% to about 10% (wt/wt) of the composition.

19. The pharmaceutical composition of claim 16, wherein the thickening agent is present in an amount of about 1% to about 3% (wt/wt) of the composition.

20. The pharmaceutical composition of claim 16, wherein the thickening agent is a polyacrylic acid.

21. The pharmaceutical composition of claim 1, further comprising a first antioxidant.

22. The pharmaceutical composition of claim 21, wherein the first antioxidant is selected from the group consisting of: citric acid, butylated hydroxytoluene, ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine.

23. The pharmaceutical composition of claim 21, wherein the first antioxidant is present in an amount of about 0.01% to about 1% (wt/wt) of the composition.

24. The pharmaceutical composition of claim 21, further comprising a second antioxidant

25. The pharmaceutical composition of claim 24, wherein the second antioxidant is selected from the group consisting of: citric acid, butylated hydroxytoluene, ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine.

26. The pharmaceutical composition of claim 24, wherein the second antioxidant is present in an amount of about 0.01% to about 1% (wt/wt) of the composition.

27. The pharmaceutical composition of claim 24, wherein the second antioxidant is butylated hydroxytoluene.

28. The pharmaceutical composition of claim 1, further comprising propylene glycol.

29. The pharmaceutical composition of claim 28, wherein the propylene glycol is present in an amount of about 1% to about 25% (wt/wt) of the composition.

30. The pharmaceutical composition of claim 28, wherein the propylene glycol is present in an amount of about 1% to about 20% (wt/wt) of the composition.

31. The pharmaceutical composition of claim 28, wherein the propylene glycol is present in an amount of about 10% to about 22% (wt/wt) of the composition.

32. A hydroalcoholic gel for the transdermal delivery of cannabidiol to a mammal, wherein the gel results from the combination of ingredients comprising:
a. about 0.1% to about 20% cannabidiol;
b. about 15% to about 95% alcohol having between 1 and 6 carbon atoms;
c. about 0.5% to about 20% diethylene glycol monoethyl ether;
d. 0% to about 85% water; and
e. a sufficient amount of thickening agent, and an optional neutralizer, to give the gel a viscosity in excess of about 1000 cps.

33. The hydroalcoholic gel of claim 32, wherein the hydroalcoholic gel, when applied to human skin mounted in a Franz cell, has an average in vitro flux of about 0.01 to about 30 nmol/cm²/h.

34. The hydroalcoholic gel of claim 32, wherein the hydroalcoholic gel, when applied to human skin mounted in a Franz cell, has an average in vitro lag time of about 30 minutes to about 15 hours.

35. A hydroalcoholic gel for of claim 32, wherein the hydroalcoholic gel, when applied to human skin mounted in a Franz cell, has an average in vitro skin deposition of about 1 to about 30 μmol/g.

36. A method of transdermally administering a pharmaceutical composition to a mammal comprising the steps of:
a. providing a pharmaceutical composition comprising:
i. cannabidiol present in an amount of about 0.1% to about 20% (wt/wt) of the composition;
ii a lower alcohol having between 1 and 6 carbon atoms present in an amount of about 15% to about 95% (wt/wt) of the composition;
iii. a first penetration enhancer present in an amount of about 0.1% to about 20% (wt/wt); and
iv. water in a quantity sufficient for the composition to total 100% (wt/wt); and
b. applying the pharmaceutical composition to the skin of a mammal.

37. A method of topically administering a pharmaceutical composition to a mammal comprising the step of:
a. providing a pharmaceutical composition comprising:
i. cannabidiol present in an amount of about 0.1% to about 20% (wt/wt) of the composition;
ii. a lower alcohol having between 1 and 6 carbon atoms present in an amount of about 15% to about 95% (wt/wt) of the composition;
iii. a first penetration enhancer present in an amount of about 0.1% to about 20% (wt/wt); and
iv. water in a quantity sufficient for the composition to total 100% (wt/wt); and
b. applying the pharmaceutical composition to the skin of a mammal.

38. A method of treating a medical condition in a mammal comprising the steps of:
a. providing a pharmaceutical composition comprising:
i. cannabidiol present in an amount of about 0.1% to about 20% (wt/wt) of the composition;
ii. a lower alcohol having between 1 and 6 carbon atoms present in an amount of about 15% to about 95% (wt/wt) of the composition;
iii. a first penetration enhancer present in an amount of about 0.1% to about 20% (wt/wt); and
iv. water in a quantity sufficient for the composition to total 100% (wt/wt); and
b. administering a therapeutically effective amount of the composition to the skin of the mammal to treat a medical condition; and
wherein the medical condition is selected from the group consisting of: nausea, vomiting, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, synovitis, juvenile rheumatoid arthritis and inhibition of hair growth, pancreatitis and alcoholism.

39. The method of treating a medical condition in a mammal of claim 38, wherein the medical condition is osteoarthritis.

40. A pharmaceutical composition prepared by the process comprising the step of: combining the following ingredients:
a. cannabidiol present in an amount of about 0.1% to about 20% (wt/wt) of the composition;
b. a lower alcohol having between 1 and 6 carbon atoms present in an amount of about 15% to about 95% (wt/wt) of the composition;
c. a first penetration enhancer present in an amount of about 0.1% to about 20% (wt/wt); and
d. water in a quantity sufficient for the composition to total 100% (wt/wt).

41. A cannabidiol gel prepared by the process comprising the step of:
combining the following amounts of ingredients per 100 grams of gel:
a. about 0.1 gram to about 20 grams of cannabidiol;
b. about 15 gram to about 95 grams of a lower alcohol having between 1 and 6 carbon atoms;
c. about 0.1 grams to about 20 grams of a first penetration enhancer;
d. about 0.1 grams to about 15 grams of a first thickening agent;
e. optionally, a neutralizing agent in an amount sufficient to form a suitable gel having a viscosity greater than about 1000 cps; and
f. water in a quantity sufficient for the composition to total 100 grams.

42. The cannabidiol gel of claim 41, wherein the neutralizing agent is selected from the group consisting of: aqueous sodium hydroxide, aqueous potassium hydroxide, aqueous ammonium hydroxide, triethanolamine, tromethamine, aminomethyl propanol, tetrahydroxypropyl ethylene diamine, diisopropanolamine, Ethomeen C-25, Di-2(ethylhexyl) amine, triamylamine, Jeffamine D-1000, b-Dimethylaminopropionitrite, Armeen CD, Alamine 7D, dodecylamine and morpholine.

43. A pharmaceutical composition comprising:
a. an active pharmaceutical agent consisting essentially of cannabidiol present in an amount of about 0.1% to about 20% (wt/wt) of the composition;
b. an alcohol present in an amount of about 15% to about 95% (wt/wt) of the composition; wherein the alcohol is selected from the group consisting of ethanol, isopropyl alcohol and mixtures of the foregoing;
c. a first penetration enhancer present in an amount of about 0.1% to about 20% (wt/wt) of the composition; wherein the first penetration enhancer is selected from the group consisting of: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, diethylene glycol monoethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes; and
d. water in a quantity sufficient for the composition to total 100% (wt/wt).

44. A method of transdermally administering a pharmaceutical composition to a mammal comprising the steps of:
a. providing a pharmaceutical composition comprising:
i. an active pharmaceutical agent consisting essentially of cannabidiol present in an amount of about 0.1% to about 20% (wt/wt) of the composition;
ii. an alcohol present in an amount of about 15% to about 95% (wt/wt) of the composition; wherein the alcohol is selected from the group consisting of ethanol, isopropyl alcohol and mixtures of the foregoing;
iii. a first penetration enhancer present in an amount of about 0.1% to about 20% (wt/wt) of the composition; wherein the first penetration enhancer is selected from the group consisting of: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, diethylene glycol monoethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes; and
iv. water in a quantity sufficient for the composition to total 100% (wt/wt); and
b. applying the pharmaceutical composition to the skin of a mammal.

45. A pharmaceutical composition comprising:
a. cannabidiol present in an amount of about 0.1% to about 20% (wt/wt) of the composition;
b. a first penetration enhancer present in an amount of about 0.1% to about 20% (wt/wt) of the composition; and
c. water in a quantity sufficient for the composition to total 100% (wt/wt).

46. A method of treating a medical condition in a mammal comprising the steps of:
a. providing a pharmaceutical composition comprising:
i. an active pharmaceutical agent consisting essentially of cannabidiol present in an amount of about 0.1% to about 20% (wt/wt) of the composition;
ii. an alcohol present in an amount of about 15% to about 95% (wt/wt) of the composition; wherein the alcohol is selected from the group consisting of ethanol, isopropyl alcohol and mixtures of the foregoing;
iii. a first penetration enhancer present in an amount of about 0.1% to about 20% (wt/wt) of the composition; wherein the first penetration enhancer is selected from the group consisting of: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, diethylene glycol monoethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes; and
iv. water in a quantity sufficient for the composition to total 100% (wt/wt); and
b. administering a therapeutically effective amount of the composition to the skin of the mammal to treat a medical condition; and
wherein the medical condition is selected from the group consisting of: nausea, vomiting, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, synovitis, juvenile rheumatoid arthritis and inhibition of hair growth, pancreatitis and alcoholism.
